(19) United States
(12) Patent Application Publication
(10) Pub. No.: US 2026/0013774 A1
(43) Pub. Date: Jan. 15, 2026

(54) MACHINE LEARNING-ENHANCED CARDIAC ELECTRONIC SKIN SYSTEM AND METHOD THEREOF

(71) Applicant: THE HONG KONG POLYTECHNIC UNIVERSITY, Hong Kong (CN)

(72) Inventors: Qiuna ZHUANG, Hong Kong (CN); Zijian ZHENG, Hong Kong (CN)

(21) Appl. No.: 19/250,241

(22) Filed: Jun. 26, 2025

Related U.S. Application Data
(60) Provisional application No. 63/670,192, filed on Jul. 12, 2024.

(51) Int. Cl. A61B 5/318; A61B 5/00; A61B 5/28

(52) U.S. Cl. CPC A61B 5/318; A61B 5/0006; A61B 5/28; A61B 5/6801

(57) ABSTRACT

A machine learning-enhanced cardiac electronic skin system comprises a breathable cardiac electronic skin (BreaCARES) device configured to be worn on a user's skin, an electronic device, and a server. The BreaCARES device comprises liquid metal (LM) circuits comprising biopotential electrodes for contacting the user's skin, a biopotential sensing chip configured to sample electrocardiogramaignals at a predetermined frequency via the biopotential electrodes, and a microcontroller unit (MCU) communicatively coupled to the biopotential sensing chip for receiving the ECG signals from the biopotential sensing chip. The electronic device is configured to receive the ECG signals from the BreaCARES device and generate real-time visual representations of the ECG signals. The server is configured to process the ECG signals using a deep neural network (DNN) trained for cardiac event classification.

### CROSS-REFERENCE TO RELATED APPLICATIONS

This application claims priority to the U.S. provisional patent application Ser. No. 63/670,192, filed Jul. 12, 2024, entitled “ICU-grade breathable cardiac electronic skin for health, diagnostics, intraoperative and postoperative monitoring”, hereby incorporated herein by reference as to its entirety.

### FIELD OF THE DISCLOSURE

The present disclosure generally relates to bioelectronics.

### BACKGROUND

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Bioelectronics combine biology and electronics to create devices or systems that interact with the body for various purposes or applications. These devices or systems may be used to monitor biological processes, deliver treatments, regenerate lost tissues, etc.

Cardiovascular diseases (CVDs) are considered as one of the leading causes of death worldwide. It has been reported to take an estimated 18 million people's lives annually. Long-term continuous monitoring of cardiac signals is crucial for the assessment of cardiovascular health, detection of acute cardiac dysfunction, and development of personalized and precision medicine system. Clinical cardiovascular monitoring platforms (such as electrocardiogramonitoring systems) provide a powerful tool for detecting abnormalities in chronic heart diseases (e.g., heart attacks, heart failure, and arrhythmias) and intensive care. However, these devices and systems are traditionally multiple hard-wired, cumbersome, and invasive, posing risks to fragile skins of the elderly, neonates, and seriously ill patients in intensive care units (ICUs).

### SUMMARY

One or more embodiments of the present disclosure provide a machine learning-enhanced cardiac electronic skin system. The system comprises a breathable cardiac electronic skin (BreaCARES) device configured to be worn on a user's skin, an electronic device, and a server. The BreaCARES device comprises liquid metal (LM) circuits comprising biopotential electrodes for contacting the user's skin, a biopotential sensing chip configured to sample electrocardiogramaignals at a predetermined frequency via the biopotential electrodes, and a microcontroller unit (MCU) communicatively coupled to the biopotential sensing chip for receiving the ECG signals from the biopotential sensing chip. The electronic device is configured to receive the ECG signals from the BreaCARES device and generate real-time visual representations of the ECG signals. The server is configured to process the ECG signals using a deep neural network (DNN) trained for cardiac event classification.

One or more embodiments of the present disclosure provide a method for monitoring cardiac activity of a user. The method comprises obtaining, by a breathable cardiac electronic skin (BreaCARES) device worn on a skin of the user, electrocardiograma (ECG) signals associated with the user, generating, on a display of an electronic device, real-time visual representations of the ECG signals, and processing, by a server, the ECG signals to classify cardiac events using a deep neural network (DNN).

Other example embodiments are discussed herein.

### BRIEF DESCRIPTION OF THE DRAWINGS

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The drawings are not to scale, unless otherwise disclosed. Certain parts of the drawings are exaggerated for explanation purposes and shall not be considered limiting unless otherwise specified.

FIG. 1 illustrates a machine learning-enhanced cardiac electronic skin system according to certain embodiments of the present disclosure.

FIG. 2 illustrates another machine learning-enhanced cardiac electronic skin system according to certain embodiments of the present disclosure.

FIG. 3A illustrates an exploded schematic of a breathable cardiac electronic skin (BreaCARES) device according to certain embodiments of the present disclosure, where micropatterned liquid metal (LM) microelectrodes are adopted as a reliable interface between the soft, rough fibre mat substrate and the rigid components. Stretchable LM vertical interconnect accesses (VIAs) are used for interlayer electrical connections for the upper and base layers of multilayered LM circuits. The dashed lines indicate the distribution and positions of the VIAs in the device.

FIG. 3B illustrates a monolithically integrated BreaCARES microelectronic system with a conformal and comfortable biointerface for wireless and real-time cardiac care through a portable device in the form of a smartphone with a customized mobile app according to certain embodiments of the present disclosure.

FIG. 3C is a block diagram of a BreaCARES device comprising electrodes, a biopotential sensing chip as the analog front end (AFE) for electrocardiogra sampling (sampling rate: 500 Hz), a 1.8V low-dropout regulator (LDO) and a 32.768 kHz crystal for driving the sampling of AFE, and a Bluetooth (BLE) integrated microcontroller unit (MCU) for wireless data acquisition, transmission, and functional control according to certain embodiments of the present disclosure.

FIG. 3D is a digital image showing functional modules of a thin, soft, high-integration-density, and battery-free BreaCARES device, which includes ECG AFE, MCU module, LDOs, BLE antenna, oscillators, rectifier and matching capacitors, and LM coil for wireless power transfer according to certain embodiments of the present disclosure.

FIG. 3E shows wireless powering of the BreaCARES device of FIG. 3D via inductance coupling according to certain embodiments of the present disclosure.

FIG. 4 are digital images showing (a) a BreaCARES device comprising various electronic components, (b) weight of the BreaCARES device, (c) thickness of the BreaCARES device at its edge; and (d) thickness of the BreaCARES device at its center according to certain embodiments of the present disclosure. The electrospun fibre mat conformally encapsulates the underlying electronic components, which is distributed along the topography of the components and largely alleviate the overall device bulkiness.

FIG. 5A is a schematic illustration showing steps of fabricating monolithically integrated BreaCARES LM microelectronic system comprising wafer-scale transfer and patterning of LM microcircuits, the printing of base-layer LM circuit and wet-adhesive biopotential electrode, mounting electronic components, and encapsulation of permeable and waterproof fibre mat according to certain embodiments of the present disclosure.

FIG. 5B is a top-view optical image of stretchable monolithic LM microcircuits including traces, contacts, coil antenna, and vertical interconnect accesses (VIAs) according to certain embodiments of the present disclosure.

FIG. 5C is the LM microcircuits of FIG. 5B in the stretched state.

FIG. 5D shows a cross-sectional scanning electron microscope (SEM) image of vertical electrical conduction by LM VIAs between upper and base LM circuit layers, and vertical electrical insulation by electrospun fibre mats according to certain embodiments of the present disclosure.

FIG. 5E shows the finite element analysis of stress distribution of LM microcircuits of FIG. 5B at a biaxial tensile strain of 150% according to certain embodiments of the present disclosure.

FIG. 5F shows impedance vs frequency of the stretchable LM coil antenna at various strains from 0 to 250% according to certain embodiments of the present disclosure.

FIG. 5G shows phase vs frequency of the stretchable LM coil antenna at various strains from 0 to 250% according to certain embodiments of the present disclosure.

FIG. 5H shows the electrical interfaces of LM microcircuits with multiple electronic components via stretchable hybrid LM solder according to certain embodiments of the present disclosure.

FIG. 5I shows electrical stability of the electronic components at the electrical interfaces of LM microcircuits under a large tensile strain of 1500% using various microresistors according to certain embodiments of the present disclosure.

FIG. 5J shows electrical stability of the electronic components at the electrical interfaces of LM microcircuits under a large tensile strain of 1500% using various microcapacitors according to certain embodiments of the present disclosure.

FIG. 6 is a schematic illustration showing the layer-by-layer processing flow of a wireless BreaCARES device according to certain embodiments of the present disclosure.

FIG. 7 shows the finite element analysis of the stress distribution of the stretchable LM coil antenna at various biaxial strains according to certain embodiments of the present disclosure.

FIG. 8 shows electrical properties of stretchable LM VIAs including (a) electrical resistance change as the function of tensile strain, and (b) electrical stability during stretch-release process of 1500% strain according to certain embodiments of the present disclosure.

FIG. 9A is a schematics illustration of a machine-learning powered BreaCARES for detection and classification of drug-induced ECG arrhythmias in a rat model according to certain embodiments of the present disclosure.

FIG. 9B is a processing flow of condition classification of cardiac events obtained with BreaCARES microelectronic system using convolutional neural network (CNN), which includes original ECG signal processing, data augmentation, dataset establishment, ResNet-50 classification, and output of results according to certain embodiments of the present disclosure.

FIG. 9C shows accuracy classification of CNN for prediction of six cardiac events including pounding, obstruction, normal state, flutter, fibrillation, and death according to certain embodiments of the present disclosure.

FIG. 9D shows the ResNet-50 classification model for cardiac events according to certain embodiments of the present disclosure.

FIG. 9E show spectrograms of six cardiac events exhibiting unique patterns in the time-frequency domain according to certain embodiments of the present disclosure.

FIG. 10A shows a digital image of a wireless BreaCARES microelectronic system for continuous on-skin ECG monitoring according to certain embodiments of the present disclosure.

FIG. 10B shows skin impedance of commercial gel electrode and adhesive LM biopotential electrode as the function of frequency according to certain embodiments of the present disclosure, which shows the skin impedance of the adhesive LM biopotential electrode is lower than that for the commercial gel electrode.

FIG. 10C shows statistical results of signal-to-noise ratio (SNR) of commercial ECG device and BreaCARES microelectronic system under various daily activities including lying, sitting, walking, and exercising according to certain embodiments of the present disclosure, which shows the BreaCARES microelectronic system (referred to as “Breath-cardiac patch”) has higher SNR.

FIG. 10D shows ECG signals of commercial device and BreaCARES system under various daily activities including lying, sitting, walking, and exercising according to certain embodiments of the present disclosure, which shows the BreaCARES system (referred to as “Breath-cardiac patch”) has higher ECG.

FIG. 10E shows continuous ECG monitoring for the BreaCARES system of FIG. 10D over nine days according to certain embodiments of the present disclosure.

FIG. 10F shows statistical results of heart rate from ECG signals of 18 volunteers at still and exercising states as the functions of their (a) sex and (b) age according to certain embodiments of the present disclosure.

FIG. 10G shows statistical results of PR intervals from ECG signals of 18 volunteers at still and exercising states as the functions of their (a) sex and (b) age according to certain embodiments of the present disclosure.

FIG. 10H shows statistical results of QRS complex from ECG signals of 18 volunteers at still and exercising states as the functions of their (a) sex and (b) age according to certain embodiments of the present disclosure.

FIG. 10I shows statistical results of QT intervals from ECG signals of 18 volunteers at still and exercising states as the functions of their (a) sex and (b) age according to certain embodiments of the present disclosure.

FIG. 11A are digital images showing (a) the use of wireless BreaCARES microelectronic system and clinical GE-MAC800 system for the early detection of ECG arrhythmias, and (b) after removing the BreaCARES and clinical GE-MAC800 system, no negative effects on the skin indicated the wearing comfort and biocompatibility of the BreaCARES, according to certain embodiments of the present disclosure.

FIG. 11B shows comparison of abnormal ECG signals of atrial flutter recorded by clinical ECG monitor and BreaCARES according to certain embodiments of the present disclosure.

FIG. 11C show six clinical hypochondriases detected by wireless BreaCARES and clinical GE-MAC800 system including (a) sinus bradycardia, (b) myocardial infarction (MI), (c) ST-elevation MI (STEMI), (d) atrial fibrillation, (e) atrial flutter, (f) premature ventricular complex (PVC) according to certain embodiments of the present disclosure.

FIG. 12 shows hematoxylin and eosin (H&E) staining images of the heart slices with and without injection of calcium chloride solution according to certain embodiments of the present disclosure.

FIG. 13 shows abnormal ECG signals of premature ventricular complex (PVC) recorded by BreaCARES microelectronic system and clinical GE-MAC800 system for the early detection according to certain embodiments of the present disclosure.

FIG. 14 shows (a) digital image of wireless BreaCARES and wired clinical device on a person's chest, (b) digital image showing the intraoperative ECG monitoring of clinical patients using a wireless BreaCARES and clinical wired ECG monitor, (c) comparison of ECG signals using wireless BreaCARES and clinical wired ECG monitor during operating, (d) continuous intraoperative ECG monitoring during valve replacement surgery including the closure of the aorta valve during operation, subsequent opening of the aorta valve, and cardiac pacing for the recovery of regular hearbeat, and (e) spectrogram of the intraoperative ECG signals at various cardiac events including the closure and opening of the aorta valve, and cardiac pacing, according to certain embodiments of the present disclosure.

FIG. 15 shows abnormal ECG signals of sinus bradycardia under cardiac pacing recorded by BreaCARES microelectronic system and commercial ECG monitor (HealForce) for preoperative diagnosis of cardiac status according to certain embodiments of the present disclosure.

FIG. 16 show digital images of skin status (a) before and (b) after removing the BreaCARES microelectronic system and clinical GE-MAC800 system from a slim patient indicating the wearing comfort and biocompatibility of the BreaCARES microelectronic system according to certain embodiments of the present disclosure.

FIG. 17 show (a) and (b) digital images showing the intraoperative ECG monitoring of clinical patients using wireless BreaCARES microelectronic system and clinical wired and bulky ECG monitor, (b) comparison of postoperative ECG signals using wireless BreaCARES microelectronic system and clinical wired ECG monitor, (d) continuous postoperative ECG monitoring of a clinical patient for 24 hours in ICU using the BreaCARES, which clearly detects various cardiac arrhythmias of the patient including ECG T-wave inversion, tachycardia, and atrial fibrillation, (e) spectrogram of the postoperative ECG signals recorded by the BreaCARES at the event of atrial fibrillation, and (f) spectrogram of the post-atrial fibrillation ECG signals recorded by the BreaCARES after drug delivery of amiodarone according to certain embodiments of the present disclosure.

FIG. 18 shows comparison of ECG signals using wireless BreaCARES microelectronic system and clinical wired ECG monitor before operating according to certain embodiments of the present disclosure.

FIG. 19 is a flowchart of a method for monitoring cardiac activity of a user according to certain embodiments of the present disclosure.

FIG. 20 is a flowchart of a method for processing the ECG signals by a server according to certain embodiments of the present disclosure.

### DETAILED DESCRIPTION

The present disclosure will now be described with reference to the following examples which should be considered in all respects as illustrative and non-restrictive.

Throughout the description and the claims, the words “comprise”, “comprising”, and the like are to be construed in an inclusive sense as opposed to an exclusive or exhaustive sense; that is to say, in the sense of “comprising, but not limited to”.

Furthermore, as used herein and unless otherwise specified, the use of the ordinal adjectives “first”, “second”, etc., to describe a common object, merely indicate that different instances of like objects are being referred to, and are not intended to imply that the objects so described must be in a given sequence, either temporally, spatially, in ranking, or in any other manner.

Example embodiments relate to machine learning-enhanced cardiac electronic skin system and method thereof.

Existing systems implement digital health technologies based on soft and wearable electronic devices and systems to enable non-invasive, on-body, and portable healthcare monitoring and modulations, extending healthcare beyond traditional clinical settings. However, the present inventors have recognized that existing wearable technologies face unresolved challenges in clinical translation and adoption, with their real-world medical application remaining in early stages. For example, the existing technologies have the following these challenges: (1) difficult to achieve medical grade accuracy with real-time and sufficient datasets for clinical decision making because of instable, dynamic, and noisy biointerfaces, and the lack of complex system-level integration; (2) poor patient/consumer compliance owing to skin intolerance of uncomfortable monitoring devices with bulky form factors and impermeable gel electrodes during long-term wearing; and (3) underlying systemic or dermatological comorbidities (e.g., advanced age or atopic dermatitis) enviably compound the risk for potential skin intolerance or injury. Existing cardiovascular wearables are difficult to provide a chronically comfortable and reliable user-device interface with medical-grade accuracy for daily wear and clinical decision making.

Example embodiments solve one or more of the technical problems associated with the existing technologies and provide technical solutions with novel designs and improved performance.

One or more embodiments provide an ICU-grade breathable cardiac electronic skin system or platform assisted with a machine learning model for real-time, wireless, continuous, and comfortable cardiac care. The system is compatible with the well-established healthcare settings at home or in clinics. The system enables the detection of acute cardiac dysfunctions and provides continuous electrocardiogramonitoring on animal models, healthy volunteers, preoperative, intraoperative, and postoperative surgical patients in intensive care units.

One or more embodiments provide a wireless, fully integrated, and breathable cardiac electronic skin (BreaCARES) device in a thin (thickness in a range from 180 μm to 1.1 mm), lightweight (0.49 g), soft (skin-like), and permeable (680 g/m²/day) design form factor for continuous and comfortable cardiac care. Multilayered permeable stretchable liquid metal (LM) microcircuits are incorporated with electrical components in a monolithic multilayered configuration, allowing the acquisition, analysis, and transmission of cardiac data in a wireless and real-time manner.

The BreaCARES device or system or platform according to one or more embodiments is low-impedance, wet-adhesive, and waterproof, allowing a stable and high-quality biointerface for continuous electrophysiological monitoring in various daily activities over weeks. No skin erythema or inflammations are observed during monitoring. By combining machine learning techniques, massive cardiac data provide a reliable diagnosis of heart diseases and disorders in the early stages. One or more embodiments have demonstrated the versatility of the BreaCARES platform on a variety of subjects, such as animal models, healthy volunteers, intraoperative surgical patients, and ICU-grade postoperative cardiac care.

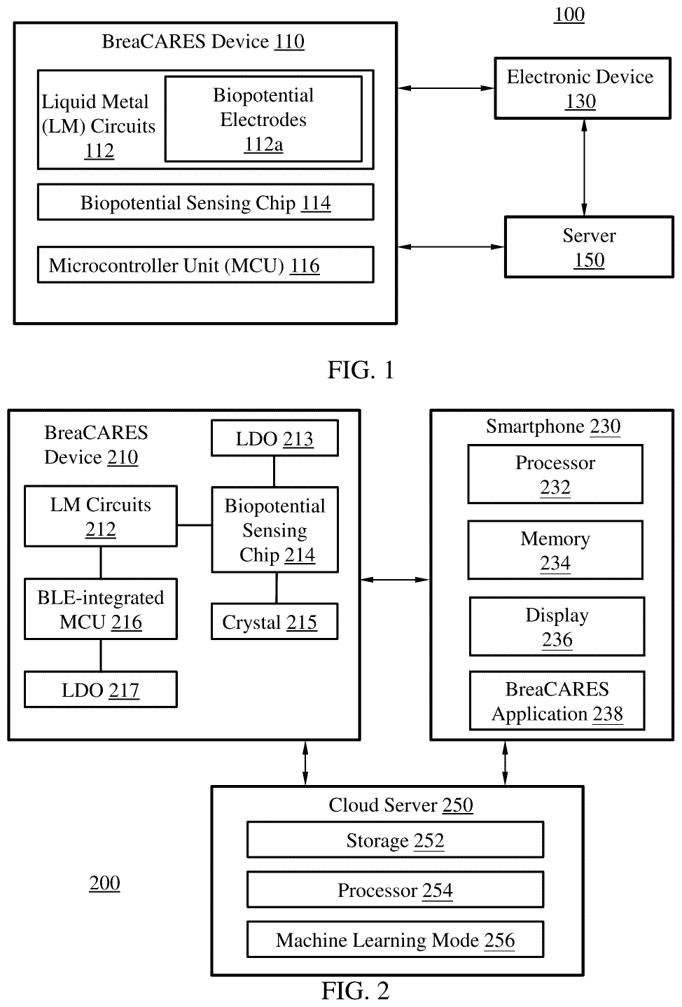

FIG. 1 illustrates a machine learning-enhanced cardiac electronic skin system 100 according to certain embodiments of the present discourse. The system 100 comprises a breathable cardiac electronic skin (BreaCARES) device 110, an electronic device 130, and a sever 150.

The BreaCARES device 110 can be worn on a user's skin, such as being attached to a user's chest, for detecting physiological signals. The BreaCARES device can be as thin as having a thickness in a range from 180 μm to 1.1 mm. The user may be a person or an animal, such as a rat. The user is not necessarily a patient. The user can be someone in good health.

The BreaCARES device 110 comprises liquid metal (LM) circuits 112, a biopotential sensing chip 114, and a microcontroller unit (MCU) 116. The LM circuits 112 comprise LM. In some embodiments, the LM comprise gallium, a gallium alloy, or a mixture thereof. In some other embodiments, the LM comprises eutectic gallium indium alloy (EGaIn), gallium indium tin alloy (GaInSn), or a mixture thereof. The LM circuits 112 form the conductive pathways, including antennas, interconnects, pads, contacts, and vertical interconnect accesses (VIAs), etc. The LM circuits 112 facilitate physical and electrical connectivity, enabling the system's flexibility, stretchability, and biocompatibility for comfortable skin contact.

As illustrated in FIG. 1, the LM circuits 112 comprise biopotential electrodes 112a for contacting the user's skin. The biopotential electrodes 112a function as specialized sensors for detecting or measuring the electrical signals generated by biological tissues, such as those produced by the heart, brain, or muscles. In some embodiments, the biopotential electrodes comprise LM and hydrogel. The hydrogel can be conductive, biocompatible, and designed to optimize skin-electrode contact. Inclusive of the hydrogel is beneficial in various aspects, such as leading to a low interfacial impedance and improved biocompatibility and skin comfort as well as improved permeability and breathability.

The biopotential sensing chip 114 may comprise one or more integrated circuits designed to detect, amplify, and process low-amplitude electrical signals (biopotentials) generated by physiological activities, such as those from the heart, brain, or muscles. In the present embodiment, the biopotential sensing chip 114 is configured to sample electrocardiogramaignals at a predetermined frequency (such as 500 Hz) via the biopotential electrodes 112a. For example, the biopotential sensing chip 114 can serve as an analog front end (AFE) for ECG sampling at a sampling rate of 500 Hz.

The MCU 116 is communicatively coupled to the biopotential sensing chip 114 for receiving the ECG signals from the biopotential sensing chip 114. The MCU 116 can be used for various functions, such as data acquisition, processing, transmission, and functional control. The MCU 116 may handle various computational tasks, such as reading ECG signals or data from the biopotential sensing chip 114 via serial peripheral interface (SPI) communication, transmitting data to an external device, and managing system operations. In some embodiments, the MCU 116 is a Bluetooth-integrated MCU or microcontroller such that the ECG signals can be transmitted to an external device via Bluetooth Low Energy. In some embodiments, The MCU 116 is mounted onto the LM circuits 112 using hybrid LM solders.

The electronic device 130 is configured to receive the ECG signals from the BreaCARES device 110 and generate real-time visual representations of the ECG signals. For example, the electronic device 130 may plot ECG data in real-time on a graphical user interface (GUI) of an application hosted therein, thereby allowing an interested party (e.g., patients, clinicians) to monitor cardiac activity visually. The electronic device 130 can be a proper electronic device, such as a smartphone, tablet, smartwatch, laptop, desktop, personal digital assistant, or other handheld computing device suitable for use in the context as described herein.

The server 150 is configured to process the ECG signals using a deep neural network (DNN) trained for cardiac event classification. The server 150 may receive the ECG signals from the electronic device 130. Alternatively, the server 150 may receive the ECG signals from the BreaCARES device 110. The server 150 may be a cloud-based server, a physical server, or other types of servers suitable for use in the context as described herein.

The server 150 performs various functions. For example, the server 150 performs a short-time Fourier transform (STFT) on the ECG signals to transform the ECG signals from time-domain signals into time-frequency representations, such as time-frequency spectrograms. The server 150 resizes the time-frequency spectrograms to obtain resized output images and applies data augmentation to the resized output images to obtain an augmented dataset. The server 150 further feeds the augmented dataset into the DNN for condition classification of cardiac events. In some embodiments, the DNN is a convolutional neural network (CNN), such as ResNet-50. In some embodiments, the CNN is trained using a Stochastic Gradient Descent with Momentum (SGDM) optimizer.

In some embodiments, the functionalities of the server are fully integrated into the BreaCARES device. That is, the BreaCARES device has a built-in artificial intelligence feature (such as a machine learning mode) and can perform on its own the functions of the server as described herein with reference to one or more embodiments.

FIG. 2 illustrates a machine learning-enhanced cardiac electronic skin system 200 according to certain embodiments of the present discourse. The system 200 can be a specific implementation of the system 100.

The system 200 comprises a BreaCARES device 210, an electronic device in the form of a smartphone 230, and a server in the form of a cloud server 250. The BreaCARES device 210 comprises LM circuits 212, a biopotential sensing chip 214, and a BLE-integrated MCU 216. The system 200 further comprises a first low-dropout regulator (LDO) or LDO 217 configured to provide power supply to the BLE-integrated MCU 216, a second LDO or LDO 213 configured to provide power supply to the biopotential sensing chip 214, and a crystal 215 electrically connected to the biopotential sensing chip 214 and configured to provide a timing reference for sampling the ECG signals.

The smartphone 230 comprises a professor 232, a memory 234, a display 236, and a BreaCARES application 238, collectively enabling seamless interaction with the BreaCARES device 210 for real-time physiological monitoring. The memory 234 stores ECG data received from BreaCARES device 210. The BreaCARES application 238, when executed by the processor 232, generates real-time visual representations of the ECG data. The display 236, such as a touchscreen interface, renders the GUI of the BreaCARES application 238, presenting real-time visual representations (such as ECG waveforms or spectrograms) for users' review. The BreaCARES application 238, developed for platforms such as Android, communicates with the MCU 216 via SPI and BLE protocols to acquire or visualize ECG data, supporting continuous monitoring in clinical and daily settings with medical-grade accuracy and user comfort.

The cloud server 250 comprises a storage 252, a processor 254, and a machine learning model 256. The storage 252 may include high-capacity solid-state drives or distributed databases, securely stores large volumes of ECG data transmitted wirelessly from the BreaCARES device 210 or the smartphone 230, facilitating analysis and data archiving for clinical and research purposes. The processor 254, such as a multi-core CPU or GPU, executes complex computations to process ECG signals. The machine learning model 256, for example, including a convolutional neural network (CNN), analyses ECG data to classify cardiac events (e.g., fibrillation, flutter, or tachycardia) with high accuracy (e.g., 98.387%), enabling early detection of abnormalities and personalized healthcare insights.

It will be understood that by those skilled in the art the description with reference to FIG. 2 is an example for illustrative purpose only. For example, the BreaCARES device 210, the smartphone 230, or the cloud server 250 may be configured differently or comprise one or more other components. One or more components may be omitted or replaced with other components. For example, the smartphone 230 may be replaced with a tablet, a desktop, or other electronic device that can be suitably used herein. The cloud server 230 may be replaced with a dedicated server, a virtual server, or other server that can be suitably used herein.

According to one or more embodiments, an example BreaCARES device can be manufactured. By way of example, once the permeable stretchable monolithic LM circuits (LM antennas, traces, connections, contacts, LM biopotential electrodes etc.) comprising permeable stretchable LM microelectrodes is fabricated, hybrid LM solder is formulated as contact pads by using oxidized LM (80° C., 16 h). Pristine LM is used as the paste connecting LM circuits and the pins of electronic components. Then the biocompatible and wet-adhesive mat is electrospun onto the back side of the base layer of LM circuits. Vertical interconnect accesses (VIAs) are formed by engineering holes to connect the LM biopotential electrodes with the skin surface. Finally, the whole monolithic microelectronic system is encapsulated with another permeable superelastic electrospun fibre mat. The example BreaCARES device adopts Bluetooth-integrated MCU (CC2640, Texas Instruments) to achieve data acquisition, transmission, and functional control. Code composer studio (CCS) is used for MCU programming. A 3.3V LDO (TPS76933, Texas Instruments) is used to provide stable power supply for the MCU. A biopotential sensing chip (MAX30003, Analog devices) is used as the AFE for ECG sampling (sampling rate: 500 Hz). A 1.8V LDO and a 32.768 kHz crystal are equipped for driving the sampling of the AFE. The ECG data are read by the MCU through SPI communication and then transmitted to a mobile device wirelessly through Bluetooth Low Energy (BLE 5.0) and plotted in real time on the GUI of an Android application (developed by Android Studio 2022.2.1). An inductive coil and capacitors that set the resonant frequency at 13.56 MHz enable wireless power supply or charging. The received radio frequency (RF) signals are rectified with a full wave bridge rectifier with smoothing capacitor. Additionally or alternatively, the whole system can be powered by one or more lithium-ion batteries.

One or more embodiments provide characterizations of the cardiac electronic skin system. The morphology of LM 3D circuits comprising fiber mat, upper and base layers of LM traces, and LM VIAs are explored or characterized using scanning electron microscopy (SEM, TESCAN VEGA3). The electrical resistance of resistors and capacitors connected with the hybrid LM solders under different strains is measured by a four-terminal method with a source meter (Keithley 2400) coupled with a customized stretching machine (Zolix). Impedance and phase of LM coil antenna are explored by an impedance analyzer (E4991B, Keysight Technologies). Moisture permeability tests are performed according to the standard E96/E96M-13 by the cup method at constant temperature (22° C.) and humidity (63%). The testing duration is set as 72 h. Skin impedance of LM biopotential electrodes and commercial gel electrodes are conducted by an electrochemical workstation (CHI 660) using the electrochemical impedance spectroscopy (EIS) technique. The distance between testing electrodes adhered to the skin surface is set as 5 cm. Impedance and phase angle as the function of frequency ranging from 1 Hz to 10,000 Hz are recorded by an alternating current (AC) sinusoid signal amplitude of 10 mV.

According to one or more embodiments, static structural mechanics of the stretchable LM coil antenna are analyzed by finite element analysis (FEA). Material mechanical parameters and output results are summarized below in Table 1.

TABLE 1
Summary of input parameters and the output results of stress
distributions in the FEA of stretchable LM antenna

| | | Average | | | | |
|---|---|---|---|---|---|---|
| | | Young's | Average | | | |
| | | modulus | Poisson's | σₘₐₓ | σ_{avg} | σₘₐₓ/ |
| Strain | Material | (Pa) | ratio | (Pa) | (Pa) | σ_{avg} |
| 0 | SBS | 10424 | 0.47 | 0 | 0 | / |
| | LM/SBS | 9841 | 0.47 | | | |
| 50% | SBS | 10424 | 0.47 | 1.62 | 4.02 | 4.02 |
| | LM/SBS | 9841 | 0.47 | E5 | E4 | |
| 100% | SBS | 10424 | 0.47 | 3.24 | 8.04 | 4.03 |
| | LM/SBS | 9841 | 0.47 | E5 | E4 | |
| 150% | SBS | 10424 | 0.47 | 4.86 | 1.21 | 4.02 |
| | LM/SBS | 9841 | 0.47 | E5 | E5 | |
| 200% | SBS | 10424 | 0.47 | 6.48 | 1.61 | 4.03 |
| | LM/SBS | 9841 | 0.47 | E5 | E5 | |

In Table 1, SBS refers to poly(styrene-block-butadiene-block-styrene). The LM coil antenna is subjected to a series of biaxial mechanical tensions ranging from 0% to 200% strains. The stress responses are collected. The ratio of the maximum stress to the average stress is adopted as the stress concentration factor, i.e., σₘₐₓ/σ_{avg}.

One or more embodiments involve experiments with animals, such as rats. The animal surgery follows the Ethical Review of Research Experiments Involving Animal Subjects (A-0664) approved by Research Committee (Animal Research Ethics Sub-Committee) of City University of Hong Kong. In certain embodiments, a healthy female Sprague Dawley (SD) rat (aged 6 weeks, ˜300 g) is first treated with gaseous light anesthesia (isoflurane, 3%), followed by deep anesthesia by intraperitoneal injection of a mixed solution of ketamine (100 mg/kg) and xylazine (10 mg/kg). Surface ECG signals are recorded using the BreaCARES wirelessly connected with a smartphone. After intraperitoneal injection of calcium chloride solution (0.1 g/mL in normal saline), abnormal ECG signals are also recorded.

One or more embodiments involve cryosection, histological staining, and observations of the heart slice. After the rat surgery, the histological status of the heart slice is studied. To protect the tissular structure, the specimen is fixed in a phosphate-buffered saline (PBS) solution with 4 wt % formaldehyde for 24 hours and transferred into 30% sucrose solution overnight, after which it is transferred to sucrose-cryo-optimal cutting temperature media compound before frozen sectioning. Then, the brain specimen is frozen at ˜80° C. and sliced with the freezing microtome (CryoStar NX70) with a block thickness of 15 μm. The hematoxylin and eosin (H&E) staining method is adopted by using the basic dye hematoxylin (BL 735A-1) for the staining of acidic cell components (i.e., nucleic acids, glycosaminoglycans, and acid glycoproteins) and the acidic dye eosin (BL 735B) for the staining of the cell cytoplasm. Specifically, the slices are first stained with hematoxylin solution for 5 min after cleaning with deionized (DI) water. Then, the slices are thoroughly rinsed in DI water, followed by differentiation in the 0.1% acetic acid and 85% ethanol in water for 10 s and rinsed again with DI water for 1 min. Next, the slices are blued in the PBS/PBS with 0.05% Tween solution for 30 s and then rinsed with DI water. After washing the blued slice in ethanol for 10 s, eosin is applied to stain the tissue for 30 s. Last, the slices are dehydrated in ethanol (95%) and permeabilized in xylene two times (5 min each), respectively. For immunohistochemical analysis, slices are observed with the invert microscope (Nikon Eclipse Ti).

One or more embodiments provide STFT procedure of ECG signals. A comprehensive batch of ECG signals is collected, representing a range of cardiac events including Death, Fibrillation, Flutter, Normal rhythm, Obstruction, and Pounding heartbeats. A sampling frequency (fₛ) of 500 Hz is selected, which is adequate for capturing certain essential details in ECG signals, as it is well above the Nyquist rate for the highest frequency components typically present in such signals. The diversity in these signals encapsulates the complexity of cardiac rhythms and their respective pathophysiological states. To analyze these ECG signals, the STFT is employed to transform the time-domain signals into a time-frequency representation, such as time-frequency spectrograms. The continuous STFT is:

$$X ( t , f ) = {\int }_{- ∞}^{∞} ω ( t - τ ) x ( τ ) {e}^{- j 2 π f τ} d τ \qquad ( 1 )$$

where ω(t−τ) is the window function, f is the frequency parameter, x (τ) is the original signal, and X is the transformed signal. Since the collected signal is always discrete, the discrete STFT is used:

$$X ( n , f ) = {\sum }_{m = - ∞}^{∞} ω ( n - m ) x ( m ) {e}^{- j 2 π f m} \qquad ( 2 )$$

This transformation allows to observe not only the presence of various frequency components but also how these components vary over time, which is beneficial when dealing with non-stationary signals such as ECG.

During STFT, a window length of 36 is utilized to balance the resolution in the time-frequency domain, with an overlap of 20 to ensure continuity and preserve information between adjacent frames. The window function is set as Hamming window:

$$ω ( n ) = {a}_{0} - ( 1 - {a}_{0} ) cos ( \frac{2 π n}{N - 1} ) \qquad ( 3 )$$

where α₀=0.53836. The number of Fast Fourier Transform points is set to 36, which guarantees proper frequency resolution. A gap of 30 is maintained between successive STFT computations to reduce computational load without sacrificing temporal resolution. Finally, the STFT output images are resized to 224×224 pixels, a dimension suitable for feeding into neural network models without significant loss of information.

One or more embodiments provide data augmentation. Biological procedures are hard to control, so samples number varies among different classes, and some of them may be insufficient to form qualified neural network input. To address these scenarios, data augmentation techniques are employed. The following description outlines the data augmentation procedure implemented to enhance the dataset of ECG signal images for cardiac event classification according to certain embodiments. To ensure each category has an adequate number of examples, a target number of 102 images per category is set. The augmentation process is applied to any category that does not meet this threshold. The augmentation is performed using MATLAB and defined by two primary transformations. Firstly, additional salt-and-pepper noises are added by a 15% noise density. Secondly, to simulate slight variations in the ECG signal that might occur due to different heart rates or machine calibration images are further scaled along the X-axis between 90% and 110% randomly of their original size. By employing these data augmentation methods, the dataset gains variety and volume, which can improve the generalization ability of a deep learning model trained on it. The augmented dataset better represents the variability encountered in real-world scenarios, thus enhancing the robustness of the subsequent classification task.

One or more embodiments provide cardiac condition recognition by deep neural network (DNN). Certain embodiments use CNN to conduct the condition classification. Once the datastore is initialized, the dataset is automatically split into two parts: 70% of the data is used for training the model, while the remaining 30% is reserved for validation. The split is randomized to ensure that both sets are representative of the overall dataset. The network may be trained using the SGDM optimizer. Training options are configured with an initial learning rate of 0.01, a maximum of 10 epochs, and a mini-batch size of 64. A shuffle operation for the training data before each epoch is deployed to prevent the network from learning the order of the training data, which improves generalization. The training procedure is conducted on an RTX 2080 GPU platform with 16G RAM and i7 intel CPU.

One or more embodiments involve volunteer and clinical trials. All procedures in the on-skin attachment of BreaCARES on the volunteers followed ethical guidelines (HSEARS20230101001), which are approved by The Hong Kong Polytechnic University. Clinical trial of BreaCARES followed ethical guidelines (KY-H-2024-020-08) in Guangdong Provincial People's Hospital of Southern Medical University. Clinical ECG monitoring devices and systems including GE-MAC800 system, and GE Solar 8000M Patient Monitor were adopted.

More example systems, devices, and methods thereof will be described below. These examples are for illustrative purpose only to facilitate understanding of the present disclosure from various perspectives.

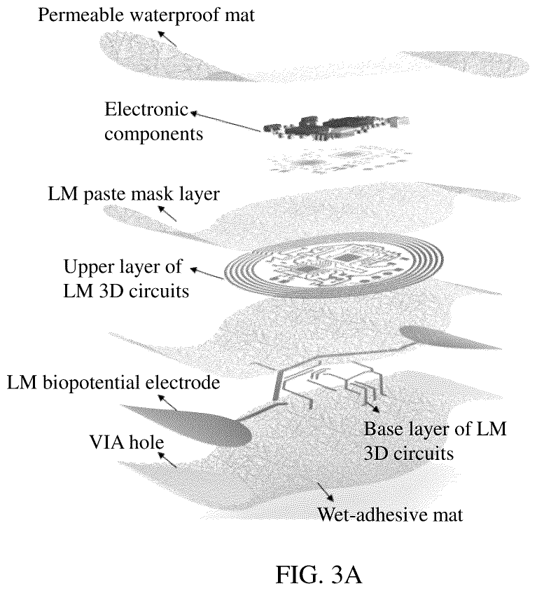

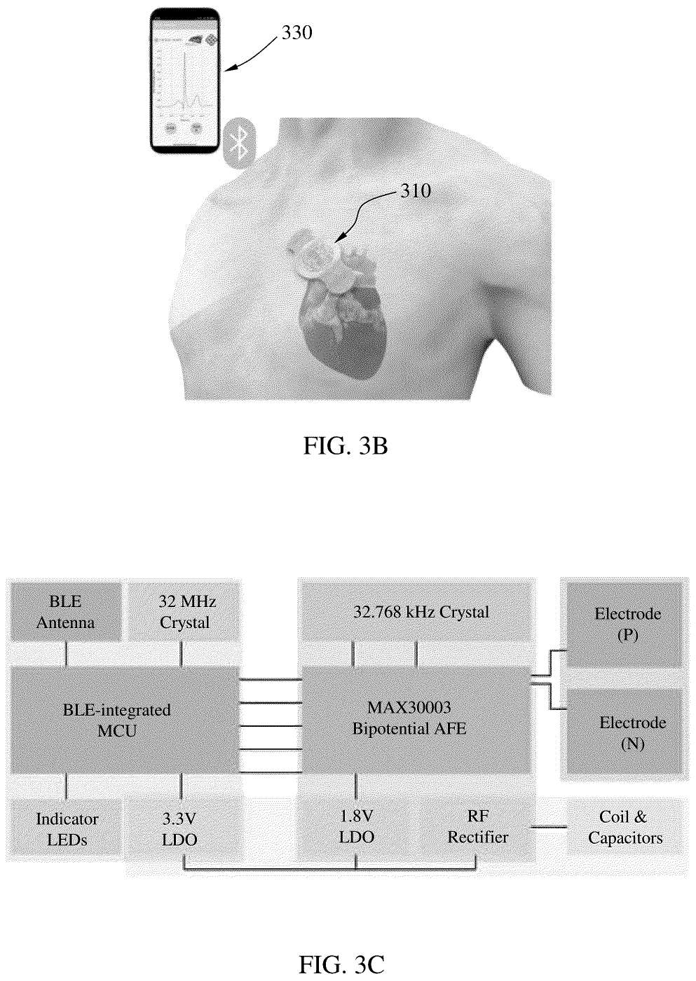

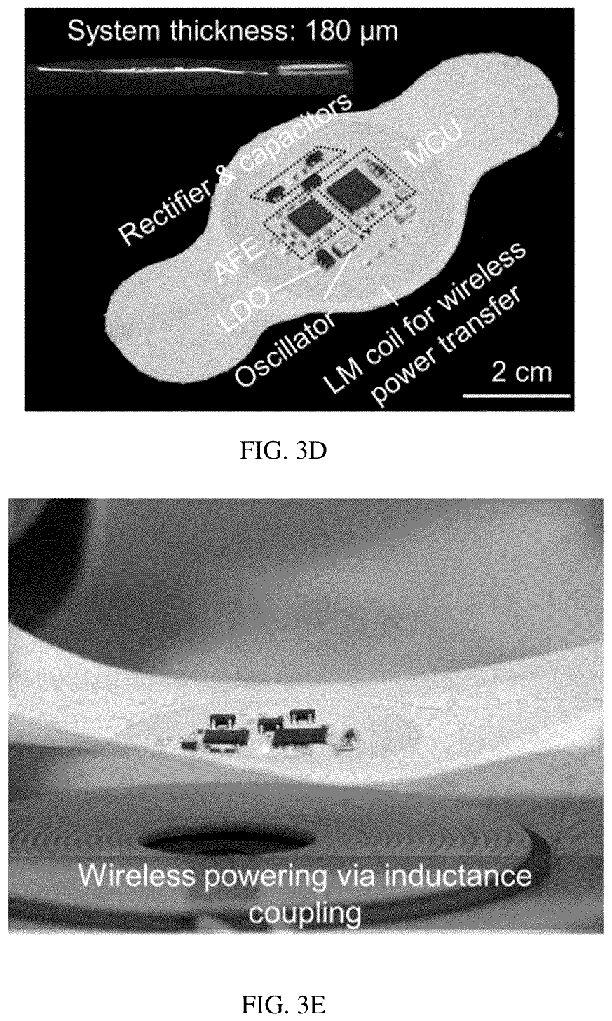

Referring to FIG. 3A to FIG. 3E, the BreaCARES device comprises five stretchable and permeable layers including a wet-adhesive mat, a LM biopotential electrode and base LM circuit layer, an upper layer of LM 3D circuit, a paste mask layer bonded with rigid electronic components, and an encapsulation layer. Eutectic gallium-based alloys are used as the LM due to their unlimited stretchability and low modulus as liquid, high electrical conductivity, and excellent biocompatibility. The micropatterned LM serves as the stretchable antenna, interconnects, pads, and contacts, while the vertical electrical connections between the base and the upper layer are achieved using LM VIAs. The LM biopotential electrode comprising LM and hydrogel enables a low interfacial impedance for low-frequency electrophysiological recording. As shown in FIG. 3B, the BreaCARES device 310 can be mounted on the user's chest, providing continuous and comfortable ECG monitoring with wireless control and communication using a smartphone 330. FIG. 3C shows that the BreaCARES device comprises a number of components, such as electrodes, a biopotential sensing chip as the analog front end (AFE) for ECG sampling at a frequency of 500 Hz, a 1.8V LDO and a 32.768 kHz crystal for driving the sampling of AFE, and a Bluetooth (BLE)-integrated microcontroller unit (MCU) for wireless data acquisition, transmission, and functional control.

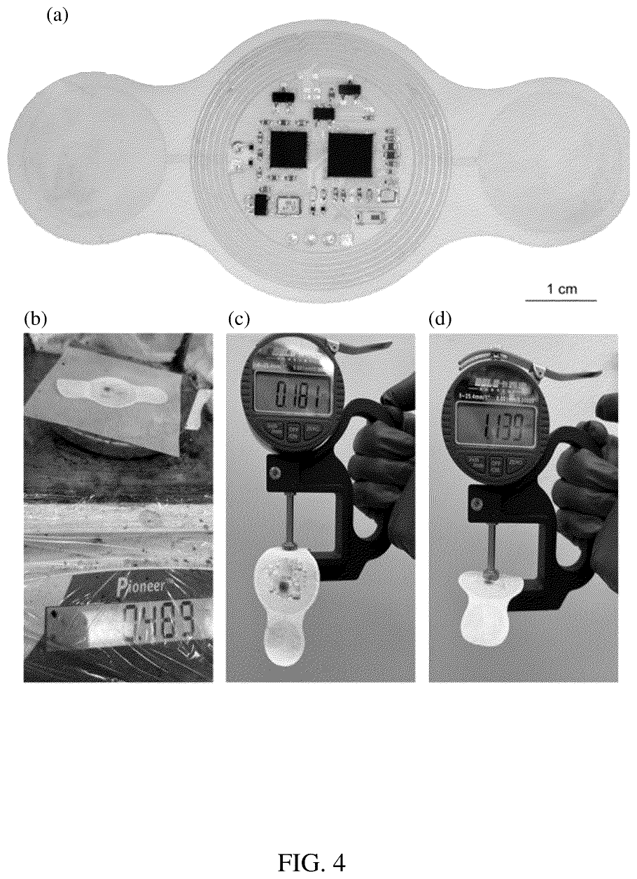

The BreaCARES device according to certain embodiments is ultrathin with a system thickness of only 181 μm (1.139 mm thickness for the rigid components), ultralightweight (0.489 g) (FIG. 4: subfigures (b)-(d)), highly stretchable and permeable, and high-integration-density (FIG. 3D). The BreaCARES device may be wirelessly powered by inductance coupling at a set resonant frequency of 13.56 MHz, enabling a fully integrated bioelectronic system for cardiac care (FIG. 3E). To the present inventors' knowledge, the proposed device or system as described herein is the most advanced cardiac system in terms of integration complexity, permeability, continuity, sampling frequency, and comprehensive clinical practice. Table 2 provides comparisons between prior art systems and the proposed system as described herein.

TABLE 2
Summary of current wearable cardiac electronic systems

| | | | | System- | | | | |
|---|---|---|---|---|---|---|---|---|
| | | | | on- | Sampling | | | |
| | Max. | Permeability | | a- | frequency | Clinical | Continuity | |
| Strategy | Strain | (g/m²/day) | Function | chip | (Hz) | practice | (h) | Ref |
| Epidermal | ~40% | N.A. | ECG | No | N.A. | No | 0.004167 | ¹ |
| electronic | | | signal | | | | | |
| skin | | | recording, | | | | | |
| | | | transmission | | | | | |
| Elastic | 500% | N.A. | ECG | No | N.A. | No | 0.0014 | ² |
| nanomembrane | | | signal | | | | | |
| | | | recording | | | | | |
| CMOS | <100% | N.A. | ECG | No | N.A. | No | 0.00138 | ³ |
| CNT | | | signal | | | | | |
| | | | recording, | | | | | |
| | | | visualization | | | | | |
| Microfluidic | 100% | Impermeable | ″ | Yes | N.A. | No | 0.0056 | ⁴ |
| assemblies | | | | | | | | |
| Binodal | 16% | Impermeable | ″ | Yes | 200 | Yes | ~0.035 | ⁵ |
| circuits/ | | | | | | | | |
| silicone | | | | | | | | |
| Transient | 45% | Impermeable | ″ | Yes | N.A. | No | 0.125 | ⁶ |
| design | | | | | | | | |
| 3D | 35% | Impermeable | ″ | Yes | N.A. | No | 4 | ⁷ |
| electronics | | | | | | | | |
| PU/PI | N.A. | Impermeable | ″ | Yes | N.A. | Yes | 24 | ⁸ |
| circuit | | | | | | | | |
| Wireless | 20% | Impermeable | ″ | Yes | N.A. | Yes | 3.5 | ⁹ |
| chest unit | | | | | | | | |
| 6-lead- | N.A. | Impermeable | ″ | Yes | N.A. | No | 0.25 | ¹⁰ |
| Au/PI | | | | | | | | |
| circuit | | | | | | | | |
| Catheter- | Flexible | Impermeable | ″ | Yes | 100 | No | 0.5 | ¹¹ |
| type | | | | | | | | |
| oximeter | | | | | | | | |
| Cu/PI | Flexible | Impermeable | ″ | Yes | N.A. | Covid | 171 | ¹² |
| circuit | | | | | | | | |
| Au/Pt/PET | Flexible | Impermeable | ″ | Yes | N.A. | No | 0.0039 | ¹³ |
| Cu/PDMS | N.A. | N.A. | ″ | Yes | 360 | No | 1 | ¹⁴ |
| Wired | Flexible | Impermeable | ″ | Yes | N.A. | No | 0.194 | ¹⁵ |
| with | | | | | | | | |
| external | | | | | | | | |
| PCB | | | | | | | | |
| BreaCARES | 750% | 680 | ″ | Yes | 500 | CP* | 198 | P* |

In Table 2, CP* refers to ICU-grade preoperative detection, intraoperative postoperative monitoring. P* refers to the proposed system according to certain embodiments of the present disclosure. Ref 1 to Ref 15 refer to the following prior art references:
1. Keum, H., Mccormick, M., Liu, P., Zhang, Y. & Omenetto, F. G. RESEARCH ARTICLES Epidermal Electronics. Science 333, (2011).
2. Jung, D. et al. Highly conductive and elastic nanomembrane for skin electronics. Science 373, 1022-026 (2021).
3. Koo, J. H. et al. Wearable Electrocardiogram Monitor Using Carbon Nanotube Electronics and Color-Tunable Organic Light-Emitting Diodes. ACS Nano 11, 10032-10041 (2017).
4. Xu, S. et al. Soft microfluidic assemblies of sensors, circuits, and radios for the skin. Science 344, 70-74 (2014).
5. Chung, H. U. et al. Binodal, wireless epidermal electronic systems with in-sensor analytics for neonatal intensive care. Science 363, 0-13 (2019).
6. Rogers, J. A. A Transient, Closed-Loop Network of Wireless, Body-Integrated Devices for Autonomous Electrotherapy. Tech. Dig.—Int. Electron Devices Meet. IEDM 2022-December 2981-2984 (2022).
7. Huang, Z. et al. Three-dimensional integrated stretchable electronics. Nat. Electron. 1, 473-480 (2018).
8. Lee, S. P. et al. Highly flexible, wearable, and disposable cardiac biosensors for remote and ambulatory monitoring. npj Digit. Med. 1, (2018).
9. Chung, H. U. et al. Skin-interfaced biosensors for advanced wireless physiological monitoring in neonatal and pediatric intensive-care units. Nat. Med. 26, 418-429 (2020).
10. Yin, L. et al. Chest-scale self-compensated epidermal electronics for standard 6-precordial-lead ECG. npj Flex. Electron. 6, 1-9 (2022).
11. Lu, W. et al. Wireless, implantable catheter-type oximeter designed for cardiac oxygen saturation. Sci. Adv. 7, (2021).
12. Jeong, H. et al. Differential cardiopulmonary monitoring system for artifact-canceled physiological tracking of athletes, workers, and COVID-19 patients. Sci. Adv. 7, (2021).
13. Zahed, M. A. et al. A Nanoporous Carbon-MXene Heterostructured Nanocomposite-Based Epidermal for Real-Time Biopotentials and Sweat Glucose Monitoring. Adv. Funct. Mater. 32, (2022).
14. Yiu, C. et al. Soft, stretchable, wireless intelligent three-lead electrocardiogra monitors with feedback functions for warning of potential heart attack. SmartMat 3, 668-684 (2022).
15. Zahed, M. A. et al. Microfluidic-Integrated Multimodal Wearable Hybrid for Wireless and Continuous Physiological Monitoring. ACS Sensors (2023), doi:10.1021/acssensors.3c00148.

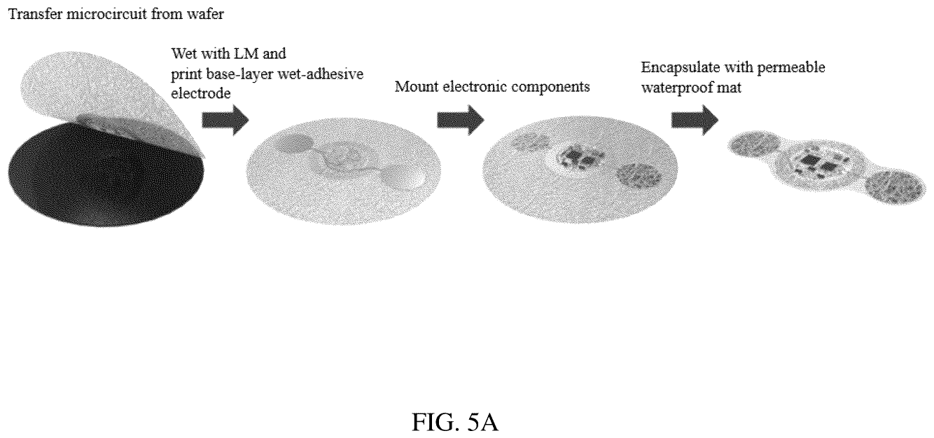

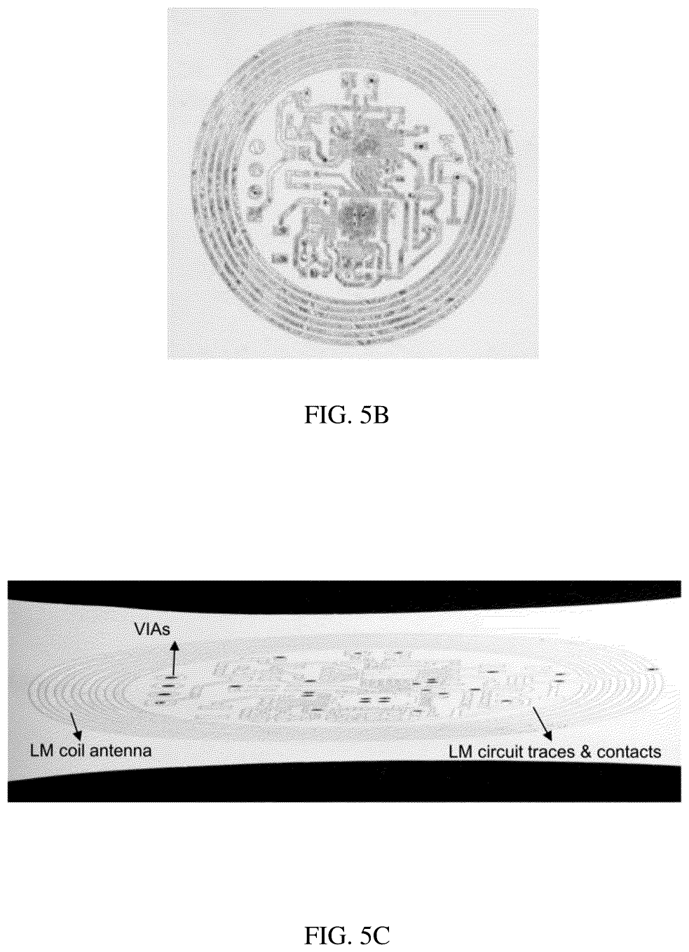

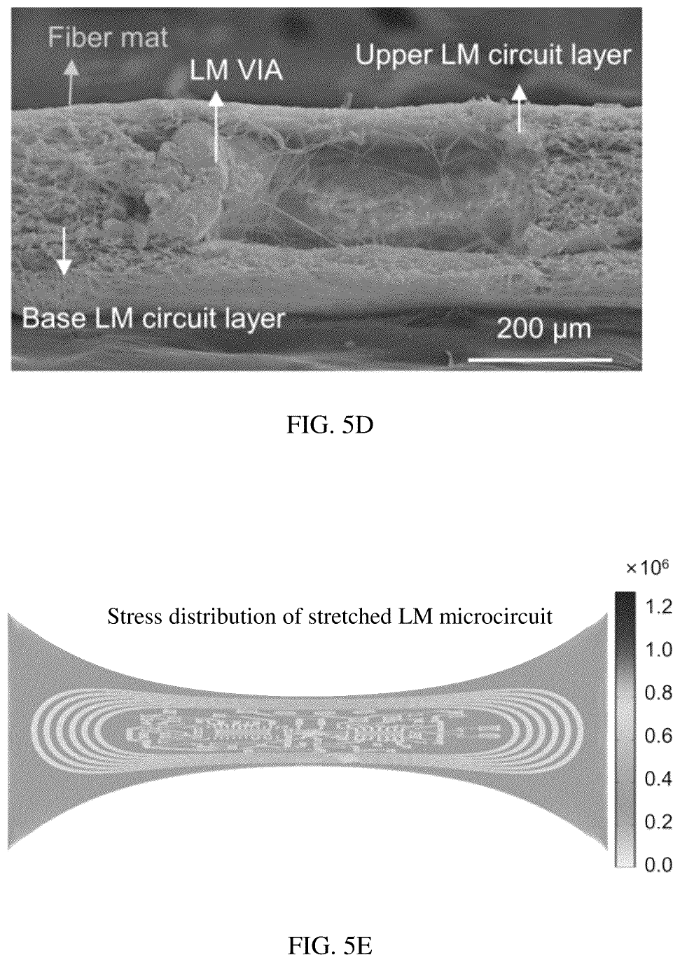

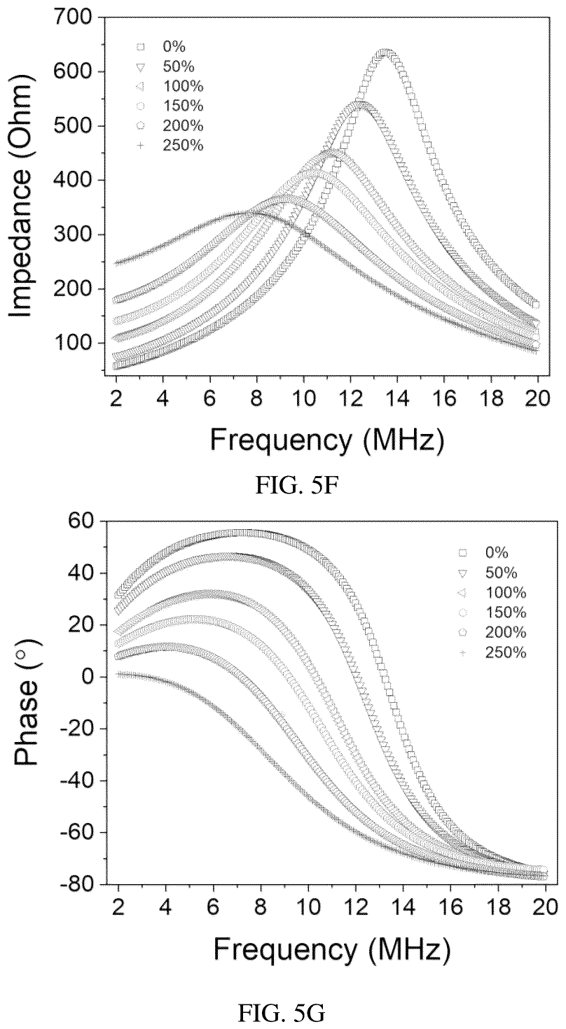

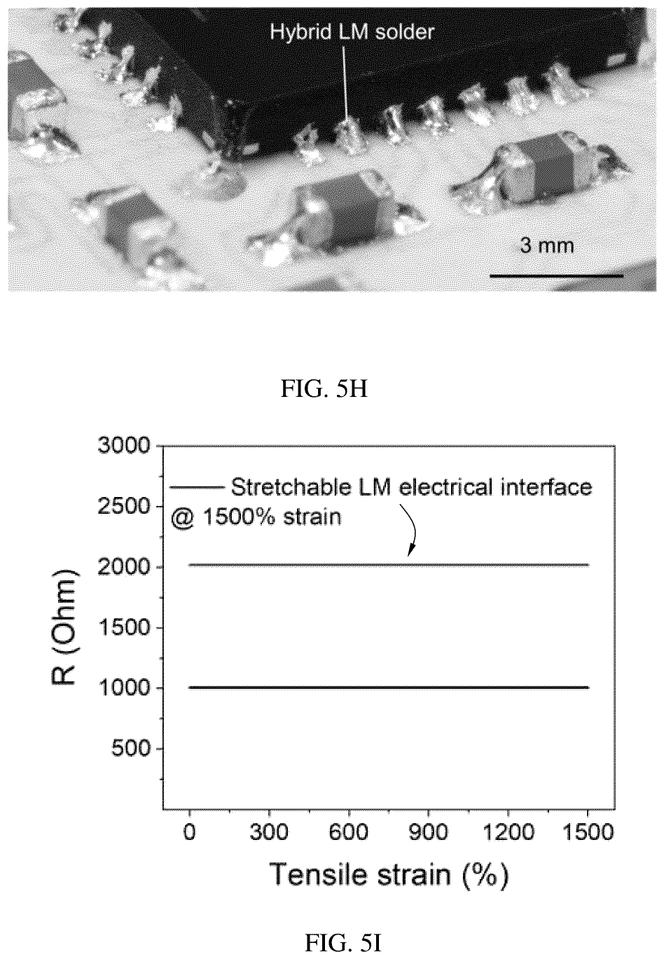

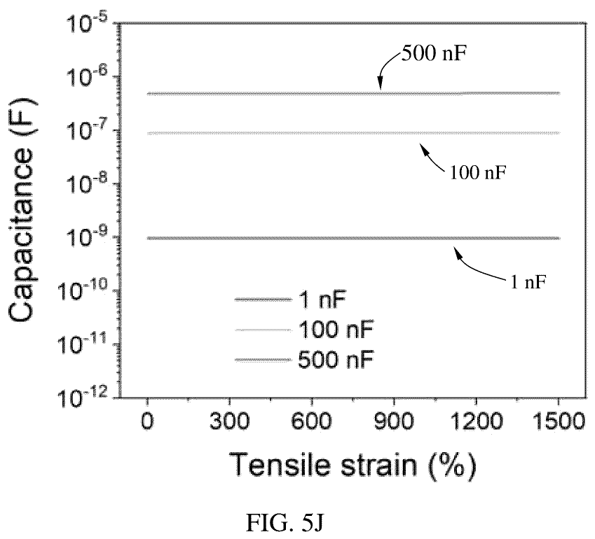

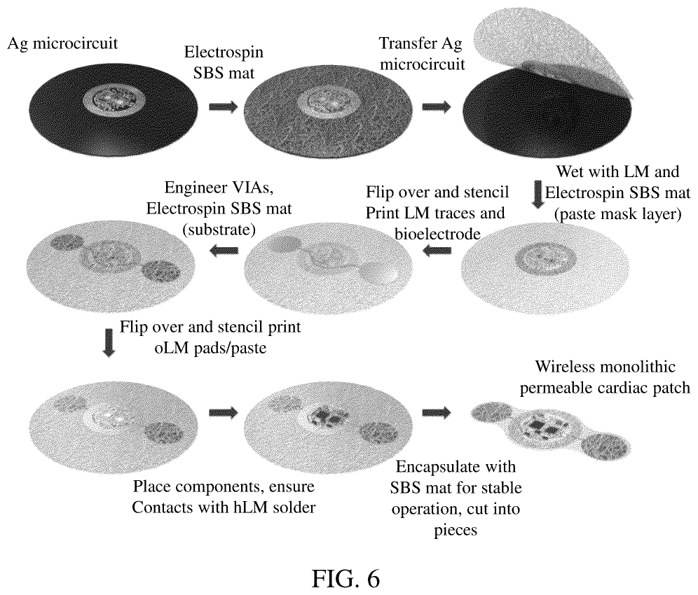

One or more embodiments provide designing of the wireless, permeable, and monolithically integrated cardiac patch. Fabrication procedures are illustrated in FIG. 5A and FIG. 6. Briefly, the base circuit layer (˜50 μm) and the upper circuit layer (˜50 μm) made of LM micropatterns on stretchable fibrous poly(styrene-block-butadiene-block-styrene) (SBS) mat are firstly fabricated by a combination of photolithography, pattern transfer, and stencil printing process. Subsequently, stencil printing of partially oxidized LM (oLM) is carried out on the paste mask layer made of thin fibrous SBS (˜30 μm), which is previously deposited on the upper circuit layer.

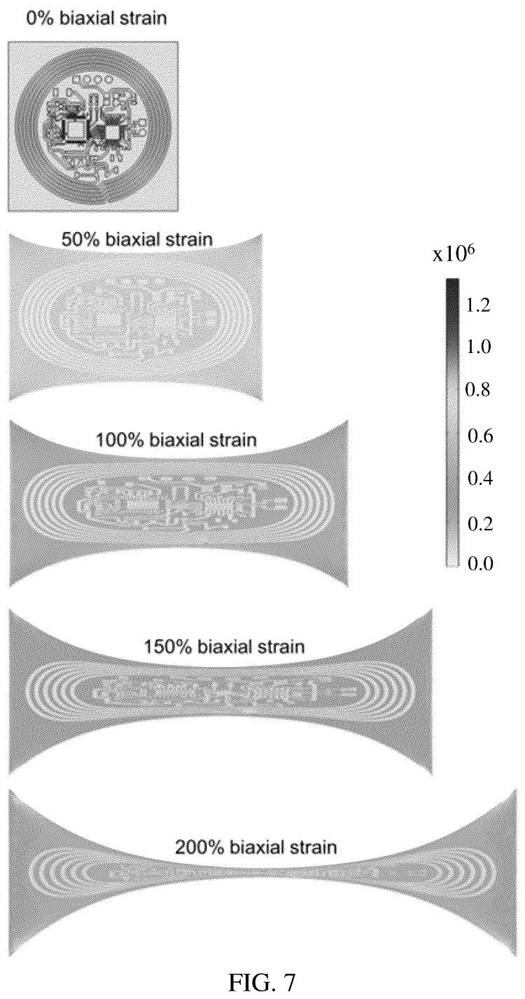

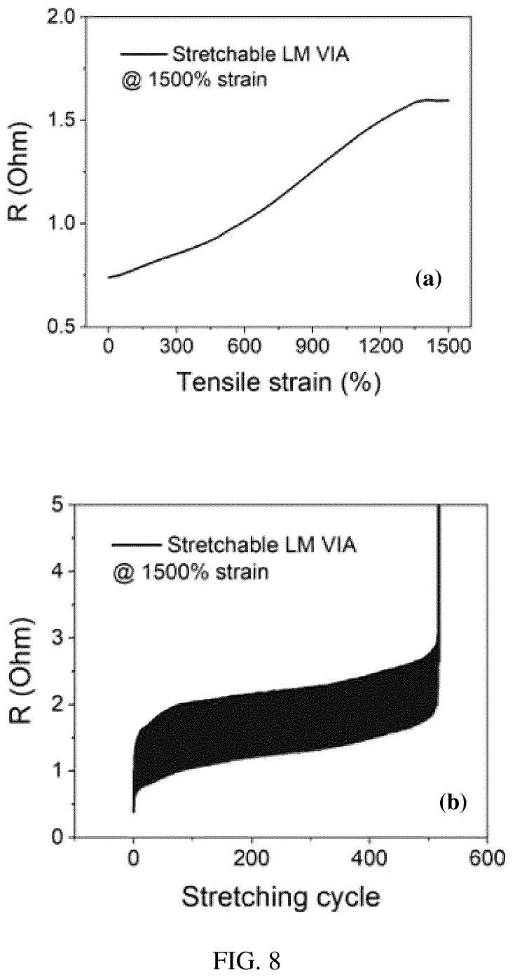

The multilayered LM microcircuits comprise stretchable antenna, interconnects, pads, contacts, and vertical electrical connections using LM VIAs (FIGS. 5B, 5C, 5D). According to the finite element analysis (FEA) results, the stress distribution of the intrinsically stretchable LM antenna is uniform under various strains (FIG. 5E and FIG. 7). In addition, the impedance and phase of the LM coil pertained to high stability under various tensile strains within the readable frequency of ˜13.56 MHZ (FIG. 5F, FIG. 5G). By formulating hybrid LM as stretchable solder (FIG. 5H), pins of rigid electronic components, including ECG AFE, light-emitting diodes (LEDs), microcontroller module (such as MCU), oscillator, BLE antenna, LDOs, and rectifier, and matching capacitors, are adhered onto the printed oLM pads with additional LM pastes. The 3D integrated electrical interfaces are highly stretchable and stable, with a maximum strain of 1500% for various electronic components (FIG. 5I, FIG. 5J, FIG. 8).

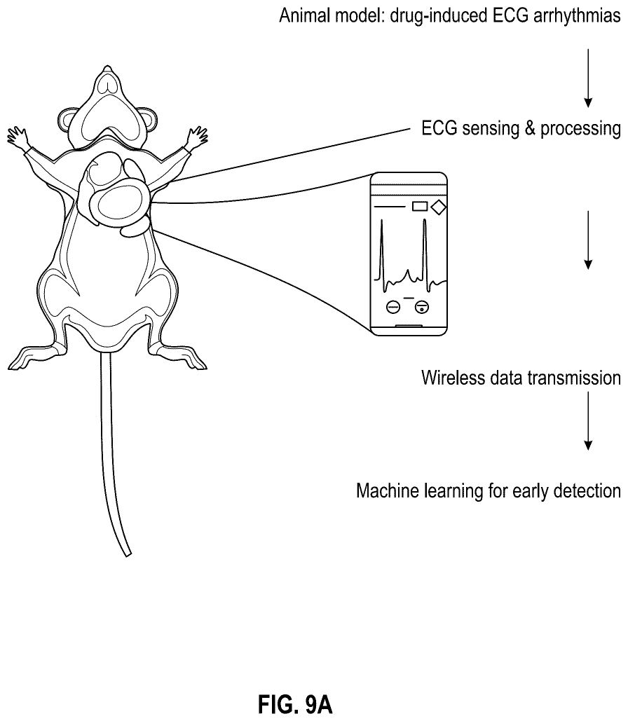

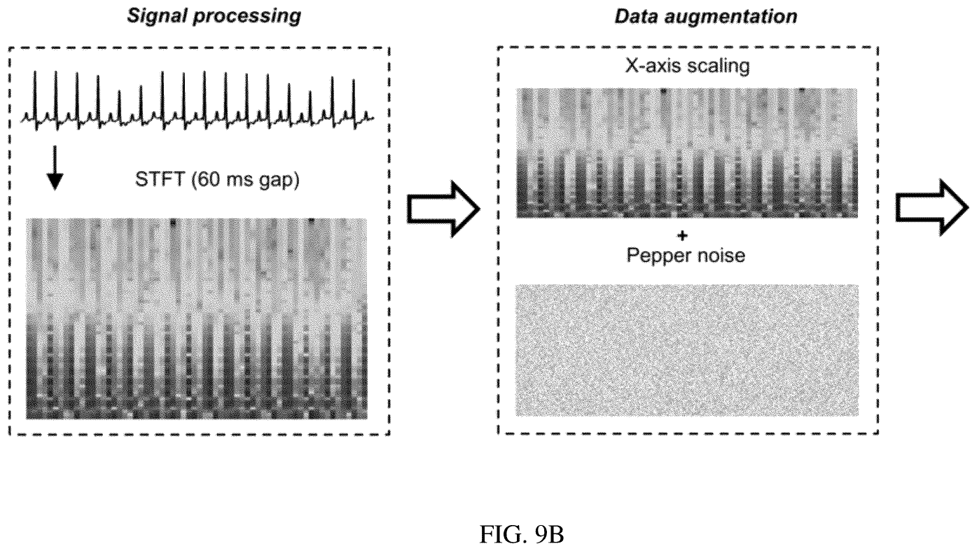

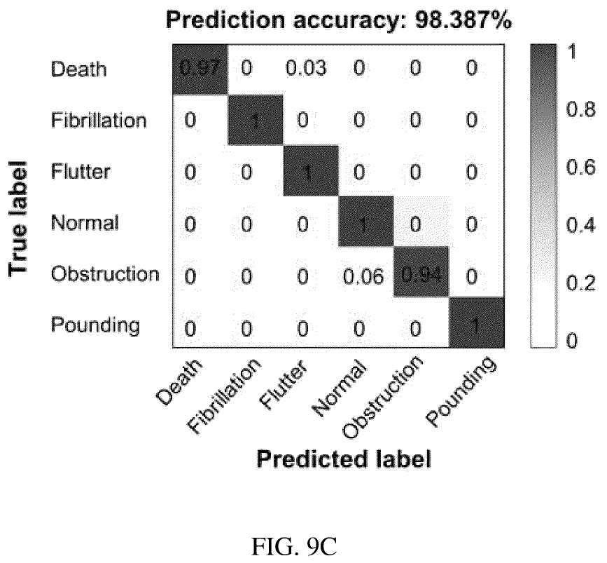

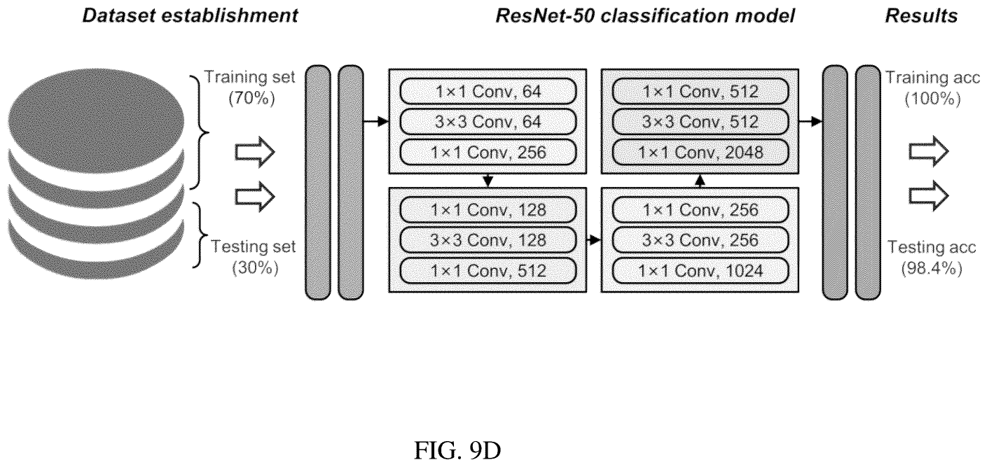

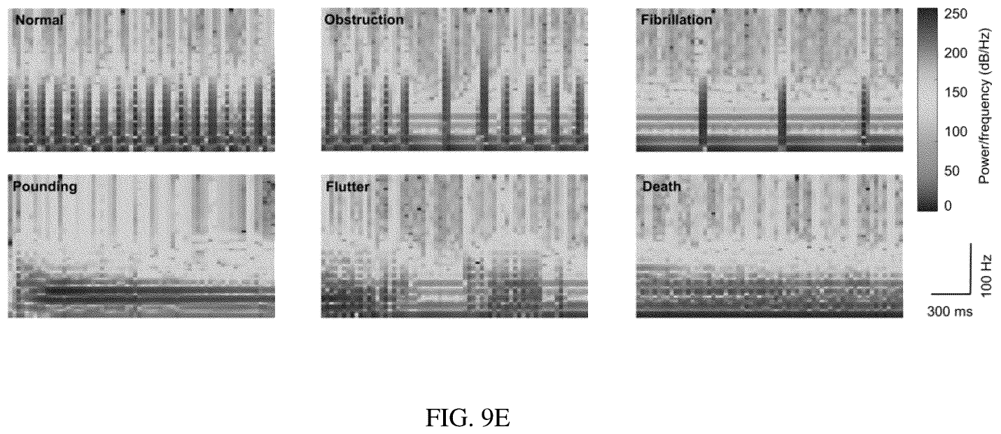

One or more embodiments provide validation in animal models. Various ECG arrhythmias of animal models are monitored using the BreaCARES device powered with machine learning algorithms. The ECG arrhythmias are induced by delivering the drug consisting of calcium chloride, which increases the levels of calcium in the blood, disrupts the heart's electrical impulses, and thus leads to abnormal heart rhythms. The ECG signals are recorded in real-time using the wireless BreaCARES device coupled with a smartphone and machine learning algorithms (FIG. 9A). The STFT is employed to transform the time-domain signals into a time-frequency representation (FIG. 9B). Further, a data augmentation procedure is implemented to enhance the dataset of ECG signal images for cardiac event classification. The CNN is adopted to conduct the condition classification of these cardiac events with a classification accuracy of 98.387% for the prediction (FIG. 9C, FIG. 9D). From FIG. 9E, the resulting spectrograms provide a clear and distinct visualization of the different types of ECG events consisting of onset of drug delivery (normal state), obstruction, fibrillation, pounding, fluttering, and the final death. These spectrograms exhibit unique patterns in the time-frequency domain, reflecting the distinct nature of each cardiac event. Right after the surgery, distinct differences in the morphology of the control sample and the drug-induced sample are found in the tissues and cells from the hematoxylin and eosin staining analysis of the heart slices.

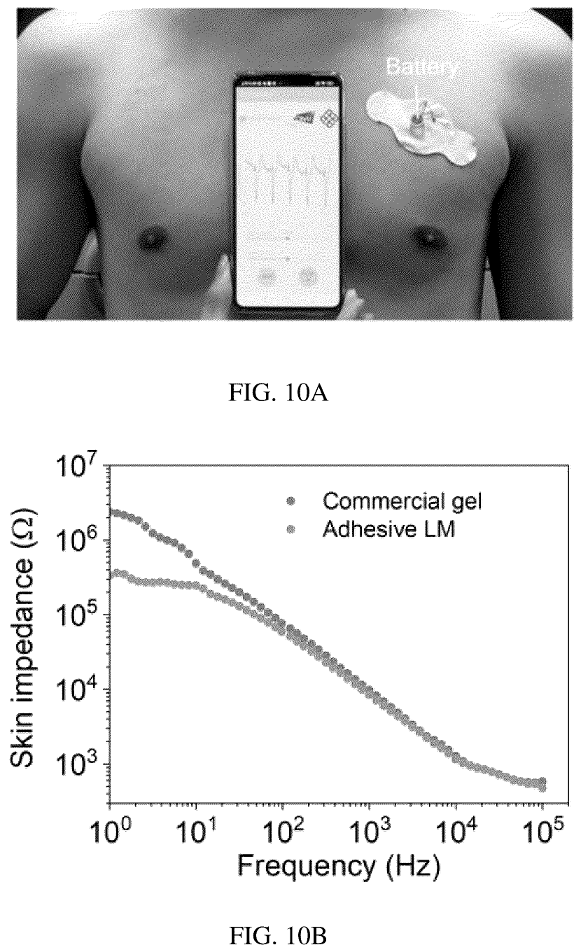

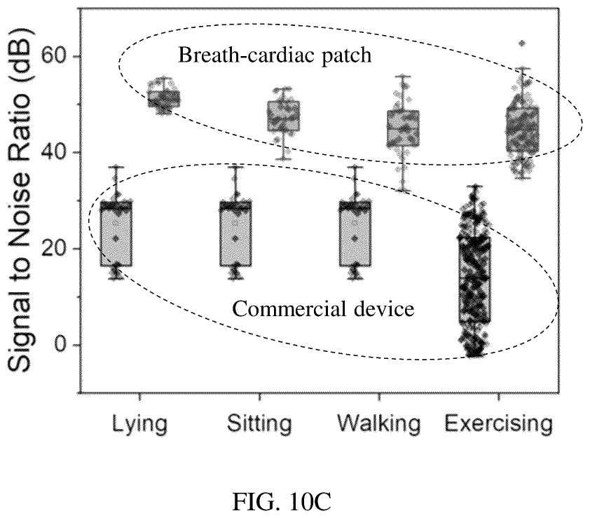

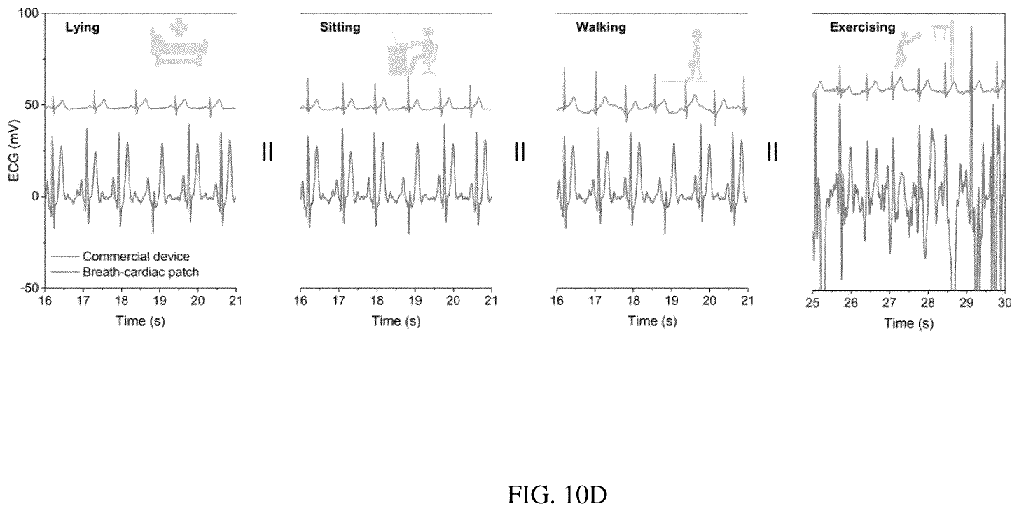

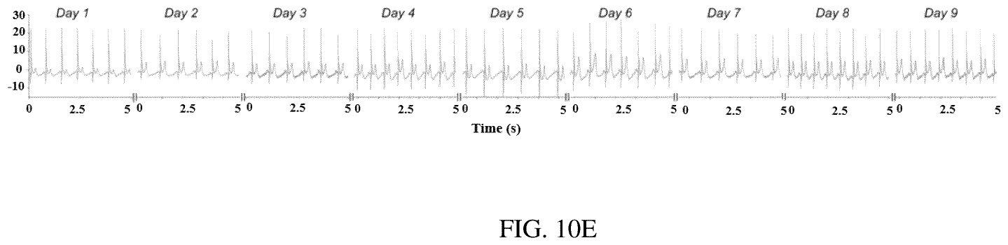

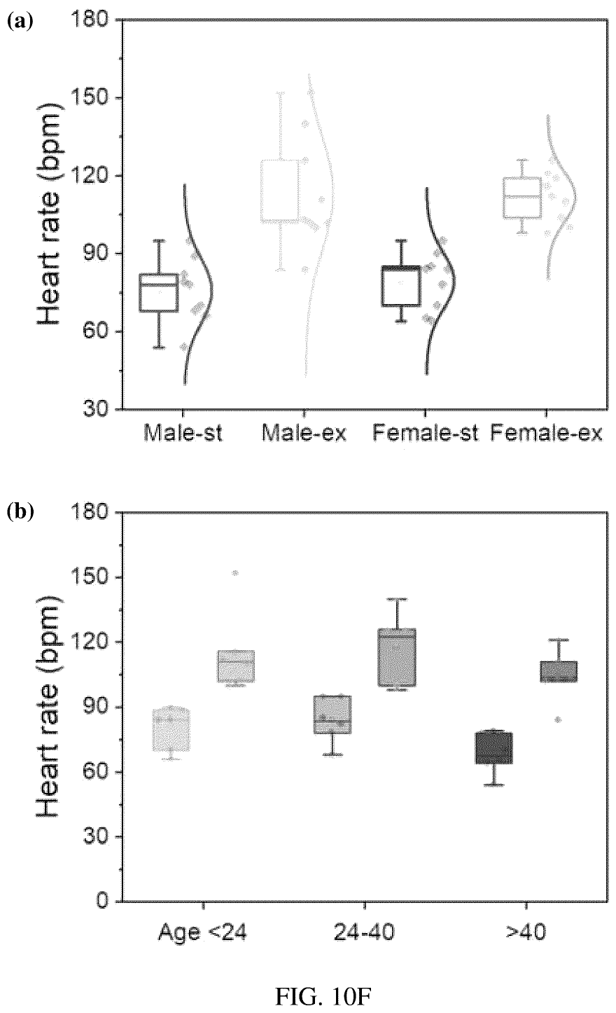

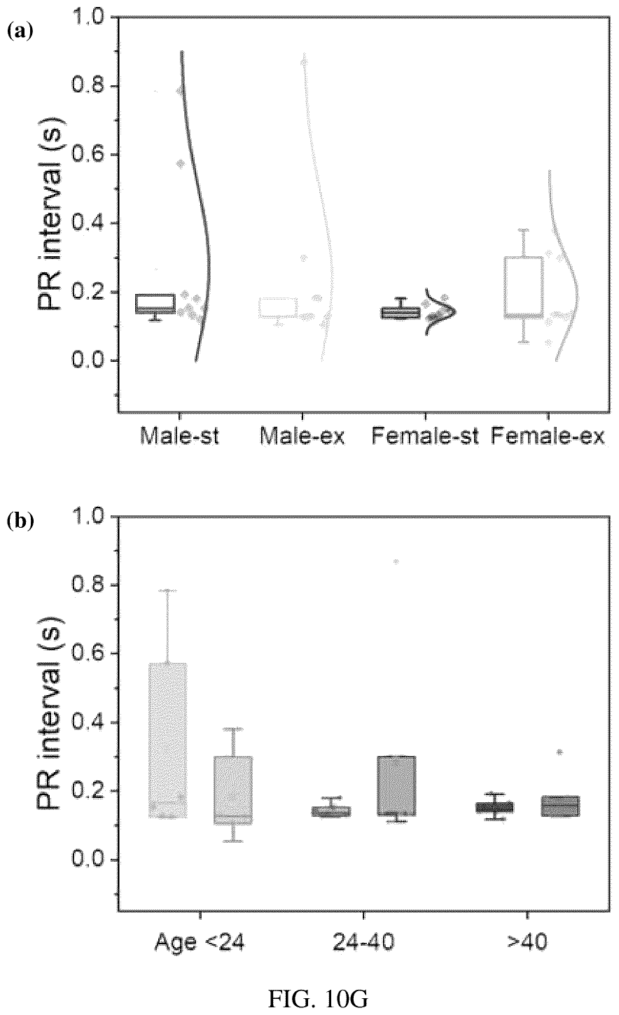

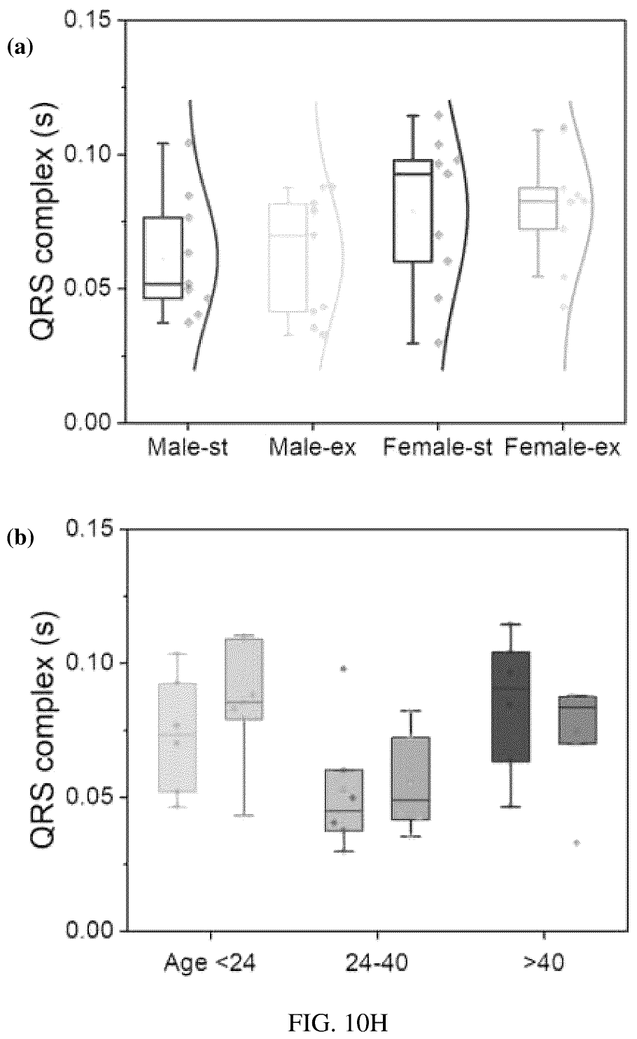

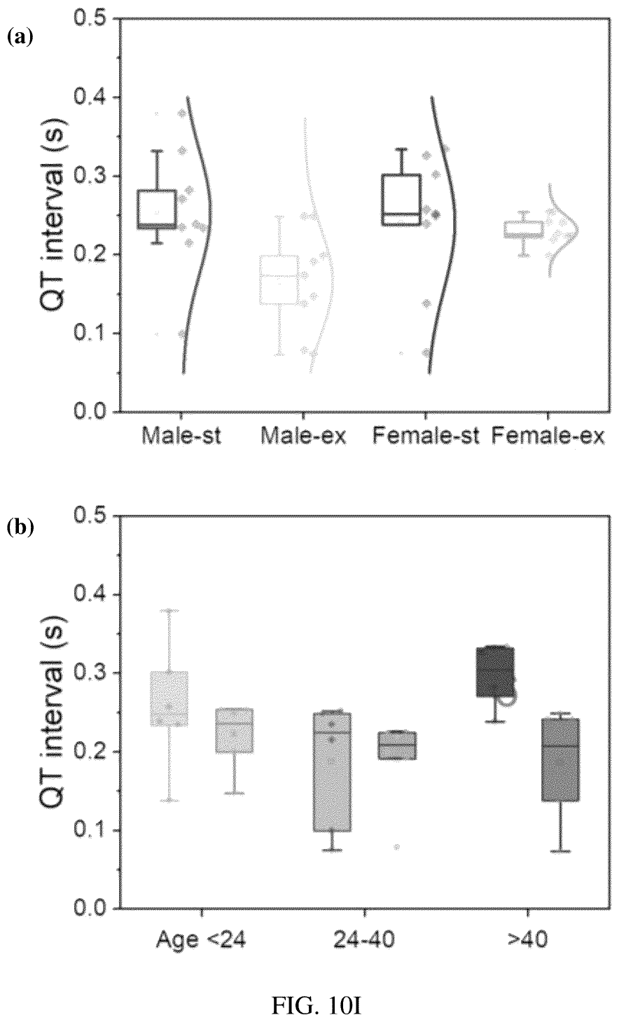

One or more embodiments provide volunteer study of BreaCARES for personalized healthcare. In comparison to the commercial wired and bulky ECG device, the BreaCARES-based device or system according to one or more embodiments offers several advantages such as portability, high signal fidelity and stability, wearing comfort, and biocompatibility to skin health. The whole cardiac electronic system is wireless and portable, enabling noninvasive, continuous, and real-time monitoring of cardiac health (FIG. 10A). The adhesive LM biopotential electrodes possess a lower skin impedance (FIG. 10B) than the commercial Ag/AgCl gels. Therefore, the skin-interfaced BreaCARES offers a more stable and higher signal fidelity during various daily activities including lying, sitting, walking, and exercising (FIG. 10C, FIG. 10D). Furthermore, the cardiac system is capable of providing a continuous and comfortable cardiac monitoring for over nine days (FIG. 10E). ECG threshold current ranges and distribution features over the whole hand exhibit large differences among populations and different statuses. The ECG signals of a testing group of 18 volunteers are monitored for different gender and age groups (<24, 24-40, and >40) under still states and exercising states (FIG. 10F to FIG. 10I). The average heart rate values under the exercising state are obviously higher than those under the still state for both males and females. The heart rate of the middle-aged (aged 24-40) group is higher than the other groups. Important intervals such as PR, QRS, and QT intervals are investigated among various age groups.

One or more embodiments provide a BreaCARES system in clinical translation, where the BreaCARES system is further applied in clinics for translational medicine in terms of early diagnosis comparable with medical apparatus, stable intraoperative monitoring, ICU-grade continuous 24-hour postoperative ECG monitoring.

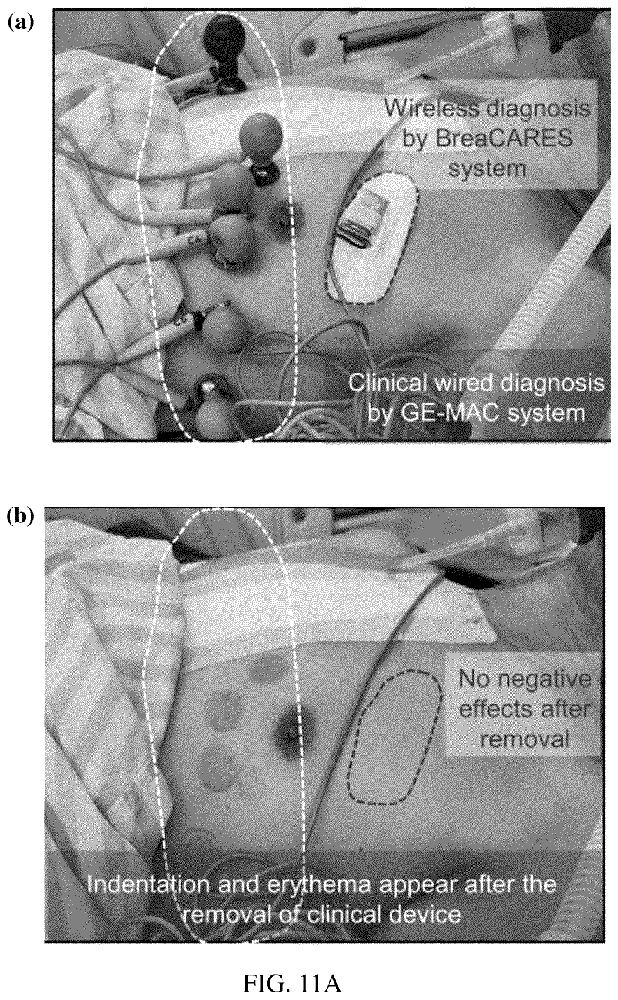

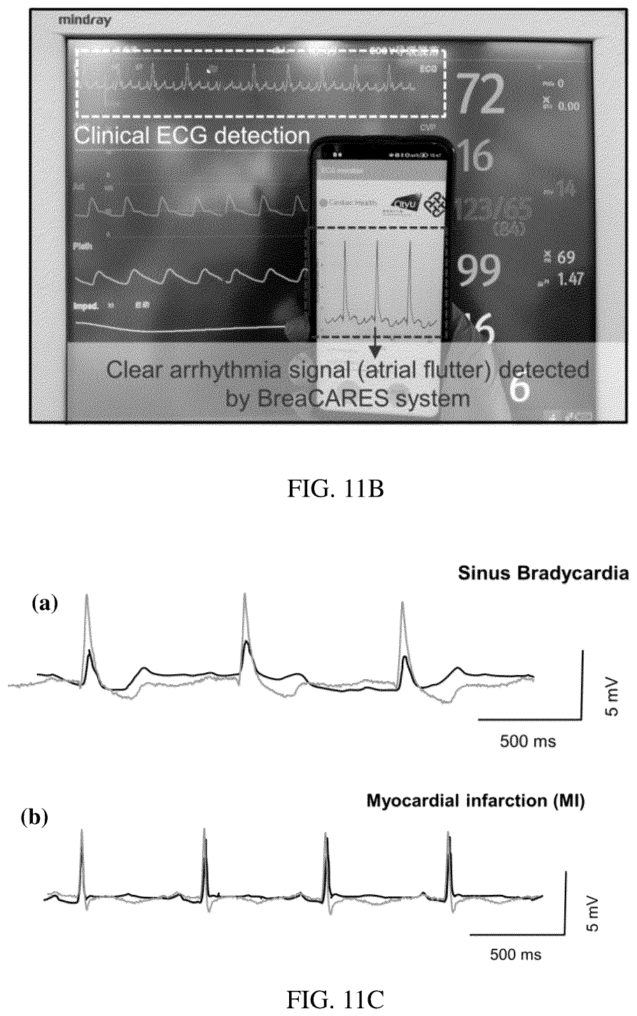

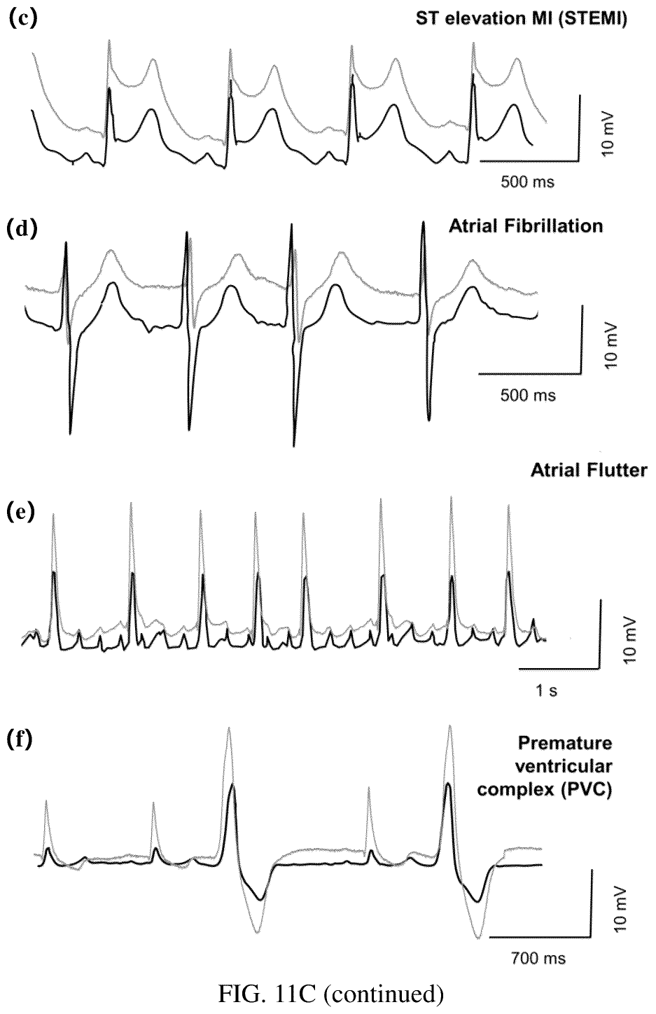

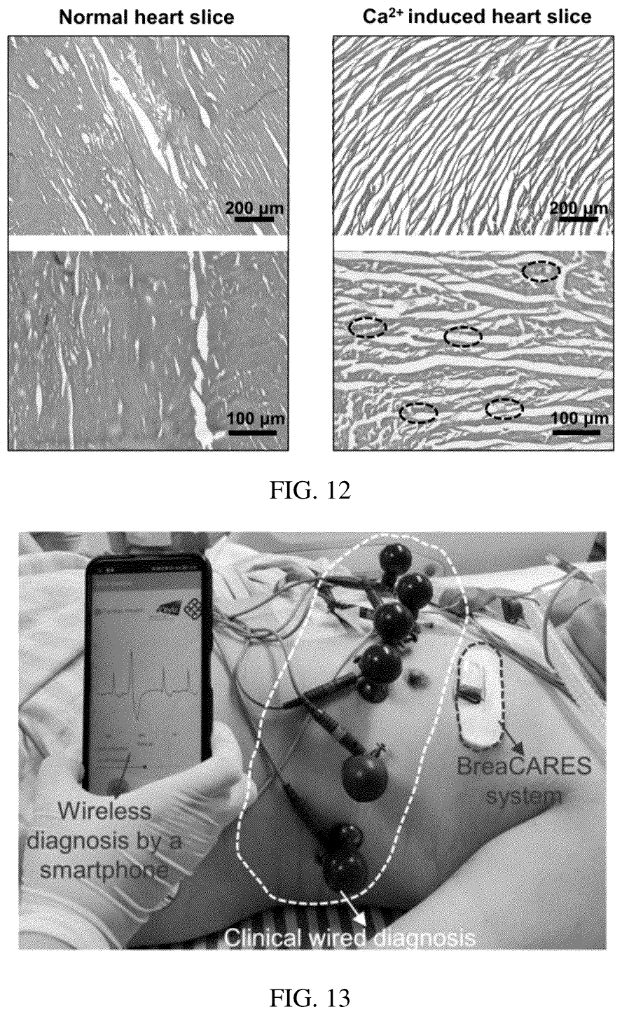

Firstly, the BreaCARES serves as a portable preoperative detection tool with medical grade reliability and superior wearing comfort (FIG. 11A). Six clinical hypochondriases including premature ventricular complex (PVC, FIG. 12), sinus bradycardia (FIG. 13), myocardial infarction (MI), ST-elevation MI (STEMI), atrial fibrillation, and atrial flutter are clearly diagnosed by the BreaCARES (FIG. 11B). The diagnostic results assisted by machine learning algorithm is 100% accurate with those of clinical diagnosis. Notably, after removing the BreaCARES, no adverse effects are caused on the skin. However, erythema and indentation result from the use of rigid clinical ECG diagnostic tool (FIG. 11C). Therefore, when involving continuous cardiac monitoring for early detection of possible cardiac diseases, the BreaCARES can offer a higher degree of wearing comfort for patients.

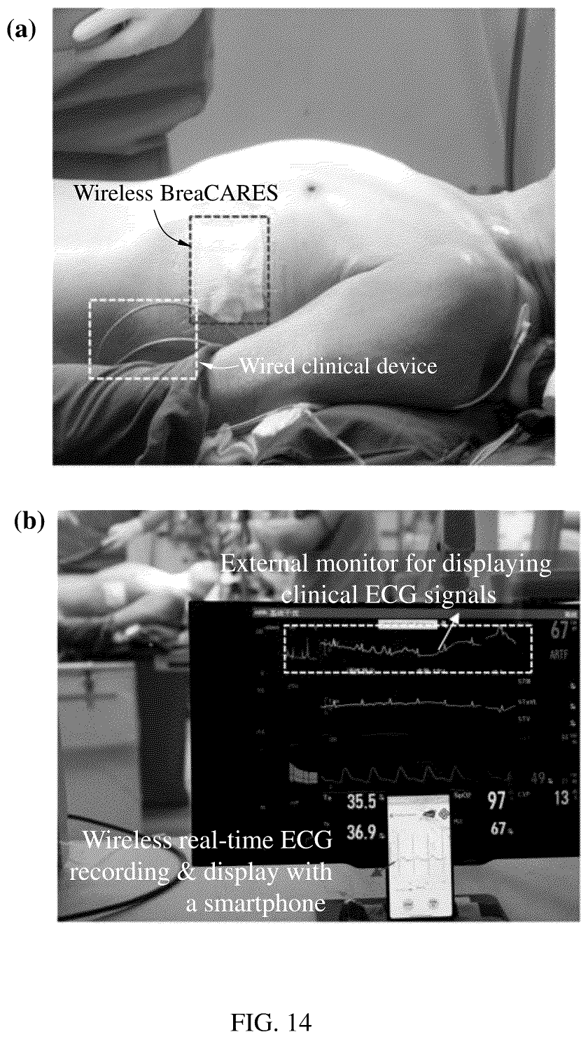

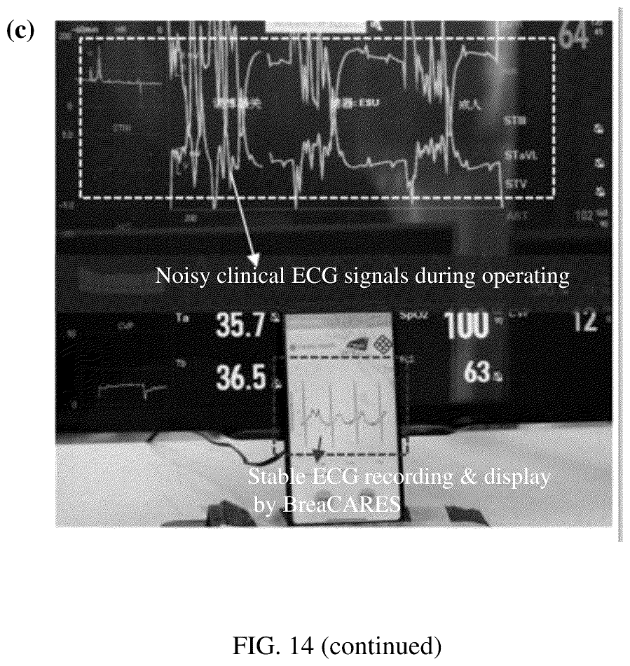

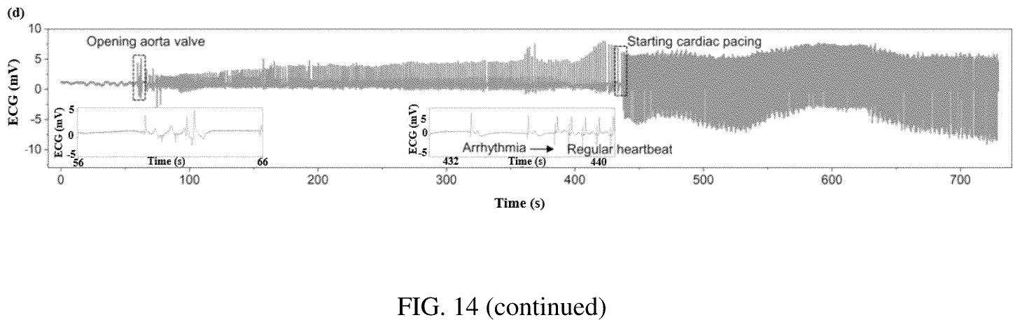

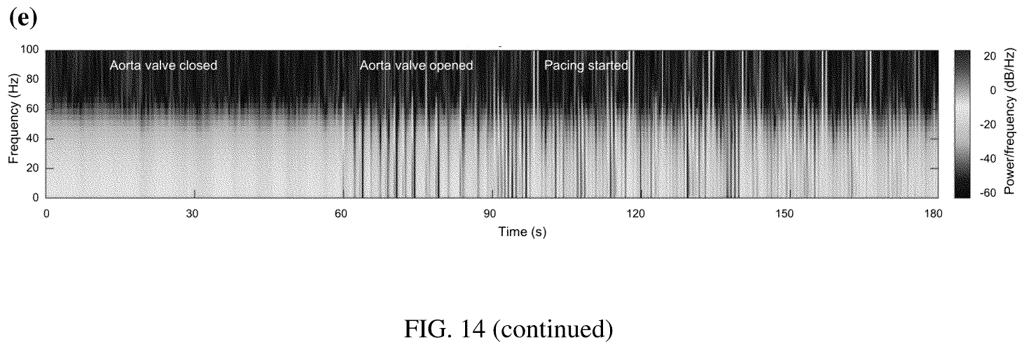

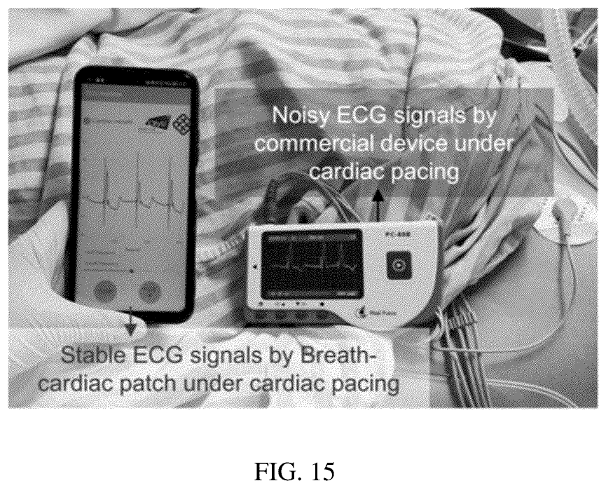

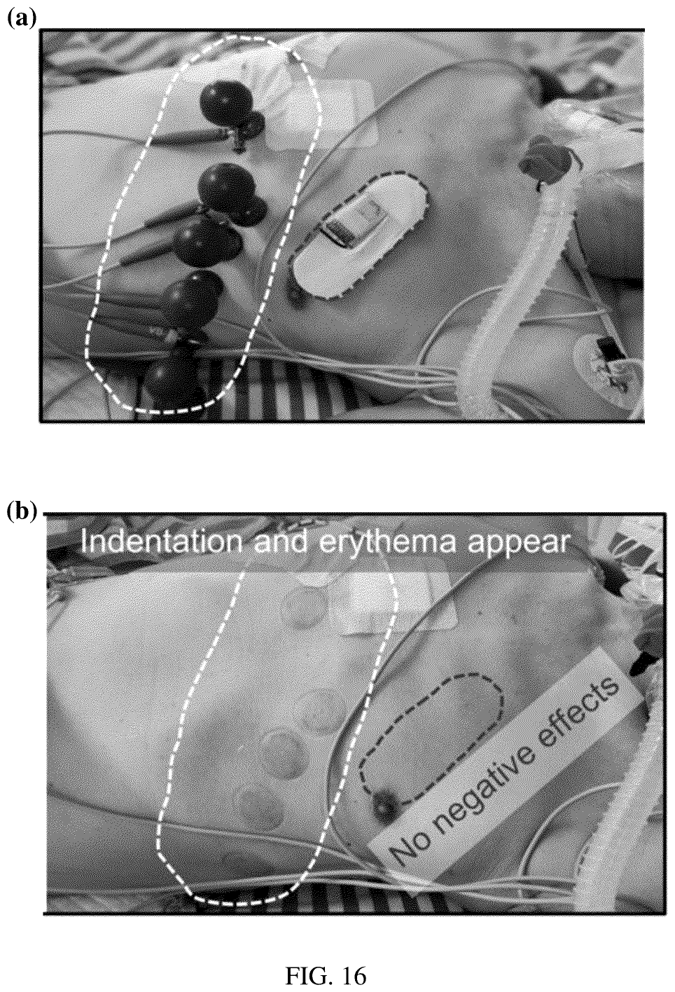

Further, the BreaCARES is adopted for intraoperative monitoring of cardiac status (FIG. 14: subfigures (a) and (b)) during the operating of mitral valve replacement (MVR) which is a surgery to replace a poorly working mitral valve with an artificial valve. During the MVR surgery, the ECG recorded by the wireless BreaCARES remains very stable either in preoperative settings (FIG. 16) or during operating (FIG. 14: subfigure (c)), while wired clinical devices display drifted and noisy signals. Various cardiac states are clearly recorded by the BreaCARES when opening the aorta valve, replacing the mitral valve, and starting cardiac pacing after the surgery (FIG. 14: subfigures (d) and (e)).

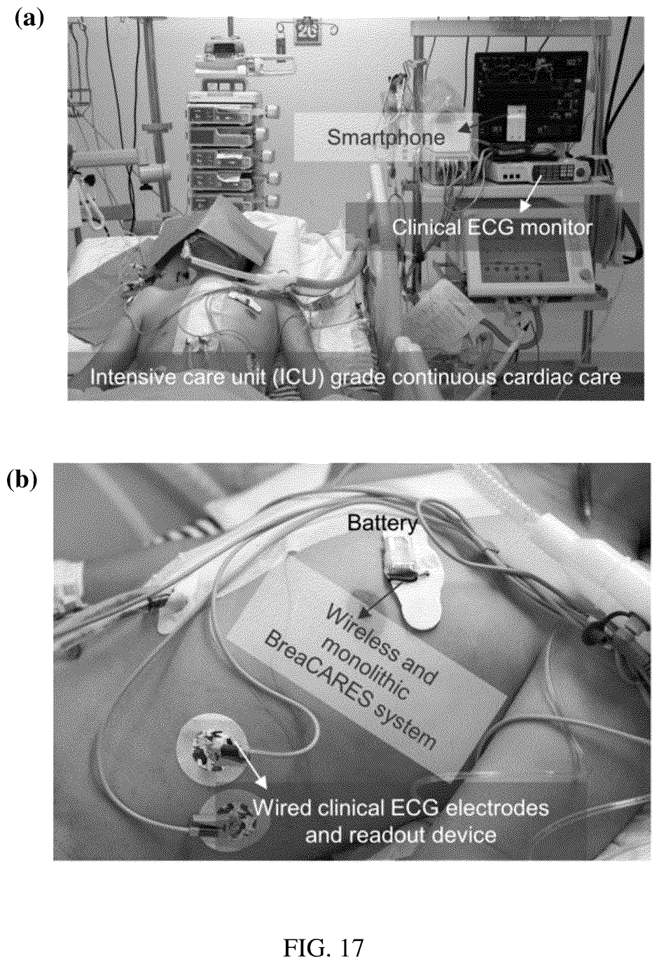

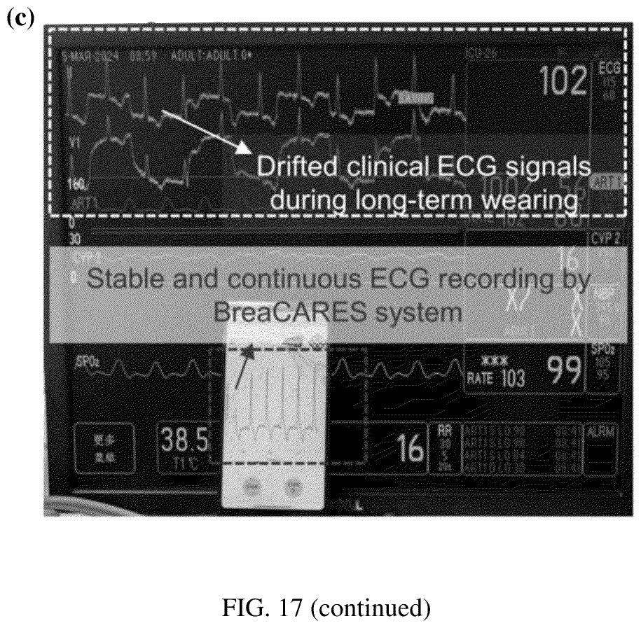

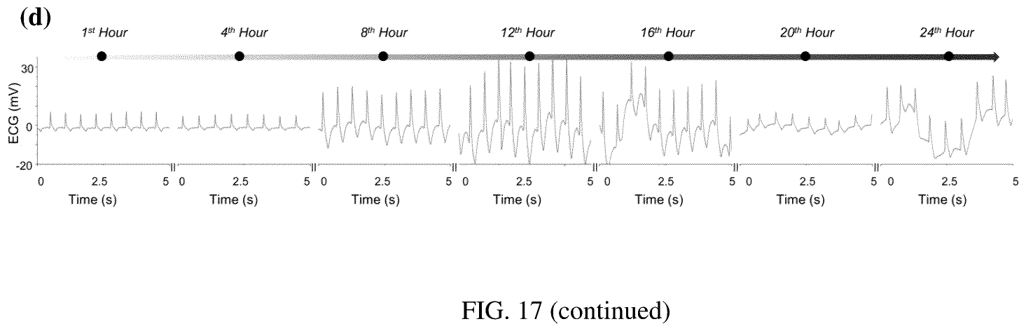

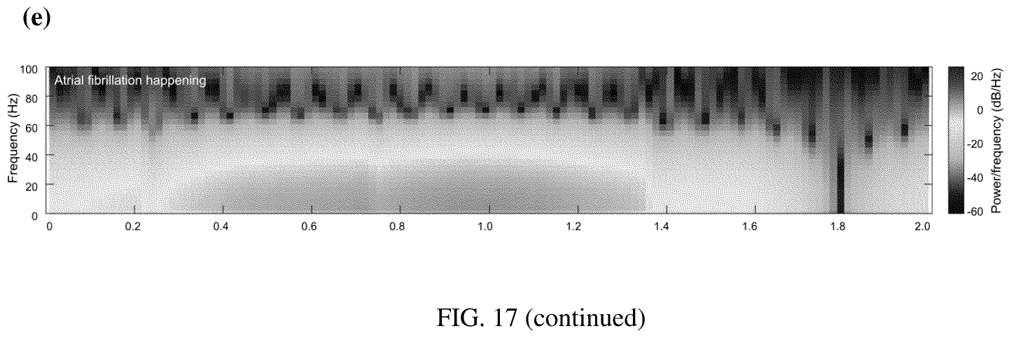

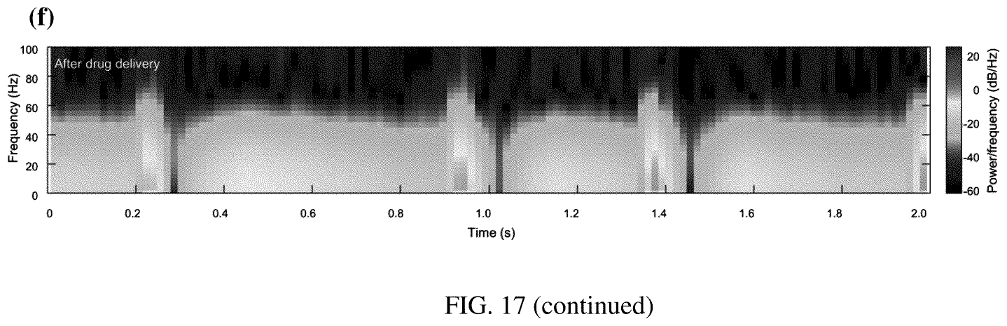

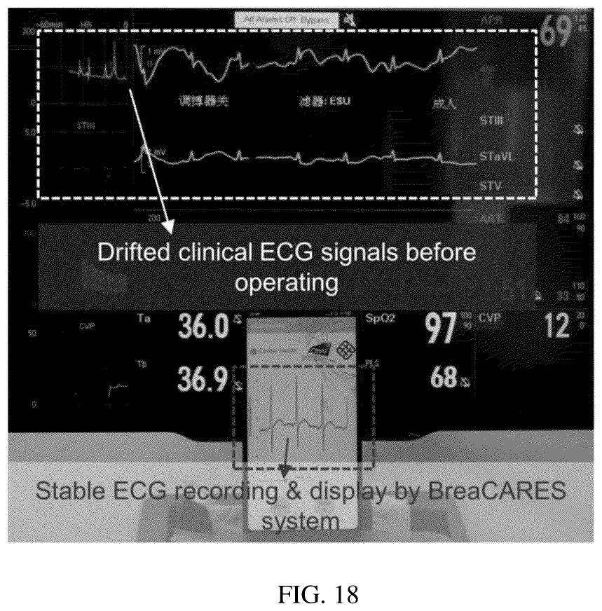

In accordance with one or more embodiments, the BreaCARES is capable of providing an ICU-grade continuous cardiac care (FIG. 17: subfigure (a)). In comparison to the wired clinical ECG monitor, the BreaCARES is more stable while avoiding the obstruction of cumbersome readout devices (FIG. 17: subfigures (b) and (c)). During the continuous ICU-grade postoperative ECG monitoring of a clinical patient for 24 hours, the BreaCARES clearly detects various cardiac arrhythmias of the patient including ECG T-wave inversion, tachycardia, and atrial fibrillation in real-time (FIG. 17: subfigures (d), (e), and (f)).

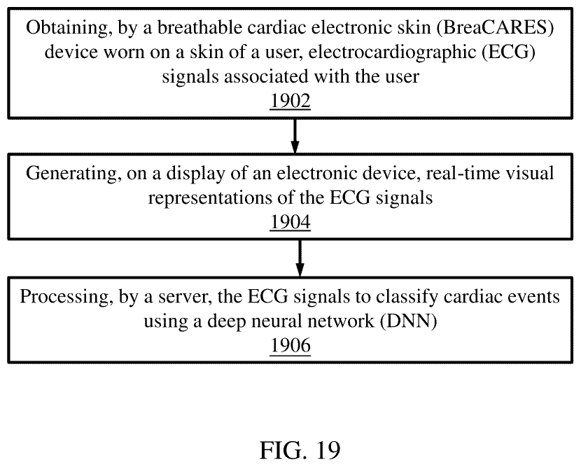

FIG. 19 is a flowchart showing a method for monitoring cardiac activity of a user. The method can be implemented by a machine learning-enhanced cardiac electronic skin system as described herein with reference to one or more embodiments.

Block 1902 states obtaining, by a breathable cardiac electronic skin (BreaCARES) device worn on a skin of the user, electrocardiogramaignals associated with the user. The BreaCARES device can be the BreaCARES device 110 or BreaCARES device 210 as described above with reference to FIG. 1 or FIG. 2 respectively. The ECG signals can be detected and acquired by the biopotential sensing chip as an AFE via the biopotential electrodes. The acquired ECG signals may be analog signals and digitalized before being transmitted to MCU for further analysis and transmission.

Block 1904 states generating, on a display of an electronic device, real-time visual representations of the ECG signals. The electronic device can be a smartphone or other proper electronic device. The smartphone plots the ECG data and displays the visualized output for review.

Block 1906 states processing, by a server, the ECG signals to classify cardiac events using a deep neural network (DNN). The sever can be a cloud server, a dedicated server, or other proper server. The server can communicate with the BreaCARES device or the electronic device via various wired or wireless channels, such as the Internet.

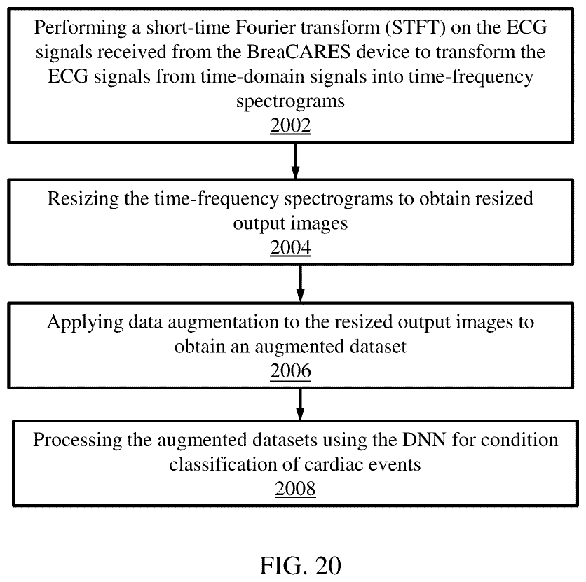

FIG. 20 is a specific implementation of Block 1906. Block 2002 states performing a short-time Fourier transform (STFT) on the ECG signals received from the BreaCARES device to transform the ECG signals from time-domain signals into time-frequency spectrograms. For example, a discrete STFT can be performed on the ECG signals at a window length of 36.

Block 2004 states resizing the time-frequency spectrograms to obtain resized output images. For example, the time-frequency spectrograms can be resized to generate the resized output images with a resolution of 224 by 224 pixels.

Block 2006 states applying data augmentation to the resized output images to obtain an augmented dataset. For example, application of the data augmentation can comprise adding salt-and-pepper noise with a noise density of 15% to the resized output images and randomly scaling the resized output images along the X-axis to between 90% and 110% of their original size to simulate variations in the ECG signals.

Block 2008 states processing the augmented datasets using the DNN for condition classification of cardiac events. For example, a CNN can be used to conduct the condition classification. The dataset can be split into a first part for model training and a second part for model validation. The CNN may be trained using a SGDM optimizer.

It will be understood the steps in the flowcharts of FIG. 19 or FIG. 20 are for illustrative purpose only. The method may comprise more steps or less steps. One or more steps may be omitted or properly modified.

As used herein, the term “server” refers to a computing system or device, whether physical or virtual, configured to provide data, services, or resources to other devices, such as portable electronic devices or wearable monitoring systems, over a wired or wireless network, including but not limited to local area networks (LAN), wide area networks (WAN), or the Internet. The server may include hardware components such as processors, memory, storage, and network interfaces, and may execute software or firmware to manage data processing, storage, transmission, or analysis, including physiological data (e.g., ECG signals) received from a monitoring system like a cardiac electronic skin. Examples of servers include, but are not limited to, cloud-based servers, dedicated servers, virtual machines, edge servers, or distributed computing systems, which may support functions such as data aggregation, real-time analytics, machine learning processing, or secure communication with client devices for health monitoring applications.

It will further be appreciated that any of the features in the above embodiments of the disclosure may be combined together and are not necessarily applied in isolation from each other. Similar combinations of two or more features from the above described embodiments or preferred forms of the disclosure can be readily made by one skilled in the art.

Unless otherwise defined, the technical and scientific terms used herein have the plain meanings as commonly understood by those skill in the art to which the example embodiments pertain. It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

What is claimed is:

1. A machine learning-enhanced cardiac electronic skin system comprising:
a breathable cardiac electronic skin (BreaCARES) device configured to be worn on a user's skin, the BreaCARES device comprising:
liquid metal (LM) circuits comprising biopotential electrodes for contacting the user's skin;
a biopotential sensing chip configured to sample electrocardiogramaignals at via the biopotential electrodes;
a microcontroller unit (MCU) communicatively coupled to the biopotential sensing chip for receiving the ECG signals from the biopotential sensing chip,
an electronic device configured to receive the ECG signals from the BreaCARES device and generate real-time visual representations of the ECG signals; and
a server configured to process the ECG signals using a deep neural network (DNN) trained for cardiac event classification.

2. The system of claim 1, wherein the server is configured to perform a short-time Fourier transform (STFT) on the ECG signals to transform the ECG signals from time-domain signals into time-frequency spectrograms.

3. The system of claim 2, wherein the server is configured to resize the time-frequency spectrograms to obtain resized output images.

4. The system of claim 3, wherein the server is configured to apply data augmentation to the resized output images to obtain an augmented dataset.

5. The system of claim 4, wherein the server is configured to feed the augmented dataset into the DNN for condition classification of cardiac events.

6. The system of claim 5, wherein the DNN is a convolutional neural network (CNN).

7. The system of claim 6, wherein the CNN is configured to be trained using a Stochastic Gradient Descent with Momentum (SGDM) optimizer.

8. The system of claim 1, wherein the electronic device is a smartphone that comprises:
a BreaCARES application configured to receive the ECG signals via Bluetooth Low Energy and generate real-time visual representations of the ECG signals; and
a display configured to display the visual representations.

9. The system of claim 1, wherein the biopotential electrodes comprise LM and hydrogel.

10. The system of claim 1, wherein the MCU is a Bluetooth-integrated MCU such that the ECG signals are transmitted to the electronic device via Bluetooth Low Energy.

11. The system of claim 10, wherein the BreaCARES device further comprises:
a first low-dropout regulator (LDO) configured to power supply to the BLE-integrated MCU;
a second LDO configured to provide power supply to the biopotential sensing chip; and
a crystal electrically connected to the biopotential sensing chip and configured to provide a timing reference for sampling the ECG signals.

12. The system of claim 1, wherein the BreaCARES device has a thickness in a range from 180 μm to 1.1 mm.

13. A method for monitoring cardiac activity of a user, the method comprising:
obtaining, by a breathable cardiac electronic skin (BreaCARES) device worn on a skin of the user, electrocardiogramaignals associated with the user;
generating, on a display of an electronic device, real-time visual representations of the ECG signals; and
processing, by a server, the ECG signals to classify cardiac events using a deep neural network (DNN).

14. The method of claim 13, wherein processing the ECG signals by the server comprises:
performing a short-time Fourier transform (STFT) on the ECG signals received from the BreaCARES device to transform the ECG signals from time-domain signals into time-frequency spectrograms;
resizing the time-frequency spectrograms to obtain resized output images;
applying data augmentation to the resized output images to obtain an augmented dataset; and
processing the augmented datasets using the DNN for condition classification of cardiac events.

15. The method of claim 14, wherein performing the STFT comprises performing a discrete STFT on the ECG signals at a window length of 36.

16. The method of claim 14, wherein resizing the time-frequency spectrograms comprises resizing the time-frequency spectrograms to generate the resized output images with a resolution of 224 by 224 pixels.

17. The method of claim 16, wherein applying data augmentation comprises:
adding salt-and-pepper noise with a noise density of 15% to the resized output images; and
randomly scaling the resized output images along the X-axis to between 90% and 110% of their original size to simulate variations in the ECG signals.

18. The method of claim 14, wherein processing the augmented datasets using the DNN comprises using a convolutional neural network (CNN) to conduct the condition classification.

19. The method of claim 18, wherein processing the augmented datasets comprises splitting the dataset into a first part for model training and a second part for model validation.

20. The method of claim 18, further comprising training the CNN using a Stochastic Gradient Descent with Momentum (SGDM) optimizer.
